(19) United States
(12) Patent Application Publication
(10) Pub. No.: US 2008/0119716 A1
(43) Pub. Date: May 22, 2008

(54) Determining presence and/or physiological motion of one or more subjects with quadrature doppler radar receiver systems

(76) Inventors: Olga Boric-Lubecke, Honolulu, HI (US); Anders Host-Madsen, Honolulu, HI (US); Victor Lubecke, Honolulu, HI (US)

Correspondence Address: MORRISON & FOERSTER LLP, 755 PAGE MILL RD, PALO ALTO, CA, 94304-1018, US

(21) Appl. No.: 11/803,654

(22) Filed: May 14, 2007

Related U.S. Application Data
(60) Provisional application No. 60/801,287, filed on May 17, 2006.
(60) Provisional application No. 60/815,529, filed on Jun. 20, 2006.
(60) Provisional application No. 60/833,705, filed on Jul. 25, 2006.
(60) Provisional application No. 60/834,369, filed on Jul. 27, 2006.
(60) Provisional application No. 60/901,463, filed on Feb. 14, 2007.
(60) Provisional application No. 60/901,415, filed on Feb. 14, 2007.
(60) Provisional application No. 60/901,416, filed on Feb. 14, 2007.
(60) Provisional application No. 60/901,417, filed on Feb. 14, 2007.
(60) Provisional application No. 60/901,498, filed on Feb. 14, 2007.
(60) Provisional application No. 60/901,354, filed on Feb. 14, 2007.
(60) Provisional application No. 60/901,464, filed on Feb. 14, 2007.

(51) Int. Cl. A61B 5/00

(52) U.S. Cl. 600/407

(57) ABSTRACT

Systems and methods for determining presence and/or physiological motion of at least one subject using a Doppler radar system having a quadrature receiver are provided. In one example, the apparatus includes a transmitter for transmitting a source signal, a quadrature receiver for receiving the source signal and a modulated source signal (e.g., as reflected from one or more subjects), and logic for mixing the source signal and the received modulated source signal to generate in-phase (I) and quadrature (Q) data, whereby nulls in the signal are avoided. In one example, the quadrature receiver further includes logic for center tracking quadrature demodulation. The apparatus may further include logic for determining physiological motion (e.g., heart rate and/or respiration rate of a person) of a subject based on the source signal and the modulated source signal.

### RELATED APPLICATIONS

The present application is related to and claims benefit of the following U.S. provisional patent applications, Ser. No. 60/833,705, filed July 25, serial no. 2006; 60/901,463, filed Feb. 14, 2007; Ser. No. 60/801,287, filed May 17, 2006; Ser. No. 60/834,369, filed Jul. 27, 2006; Ser. No. 60/815,529, filed Jun. 20, 2006; Ser. No. 60/901,415, filed Feb. 14, 2007; Ser. No. 60/901,416, filed Feb. 14, 2007; Ser. No. 60/901,417, filed Feb. 14, 2007; Ser. No. 60/901,498, filed Feb. 14, 2007; Ser. No. 60/901,354, filed Feb. 14, 2007; and Ser. No. 60/901,464, filed Feb. 14, 2007; all of which are hereby incorporated by reference as if fully set forth herein.

### STATEMENT REGARDING FEDERALLY SPONSORED RESEARCH OR DEVELOPMENT

Certain aspects described herein were made, at least in part, during work supported by a National Science Foundation grant under contract ECS0428975. The government may have certain rights in certain aspects of the invention.

### BACKGROUND

1. Field

The present invention relates generally to systems and methods for determining presence and/or physiological motion with Doppler radar, and in one example, to systems and methods for detecting the presence and/or physiological motion of zero, one, or more subjects using at least one source signal and multiple receivers.

2. Related Art

The use of Doppler radar for detection of physiological motion, e.g., related to respiratory rate and heart rate, is known. One advantage of such a method is that subjects can be monitored at a distance, without contact. Through the Doppler effect an electromagnetic wave (e.g., an RF wave) reflected at a moving surface undergoes a frequency shift proportional to the surface velocity. If the surface is moving periodically, such as the chest of person breathing, this can be characterized as a phase shift proportional to the surface displacement. If the movement is small compared to the wavelength, e.g., when measuring chest surface motion related to heart activity, a circuit that couples both the transmitted and reflected waves to a mixer for comparison produces an output signal with a low-frequency component that is directly proportional to the movement such that the heart rate can be derived.

Commercially available waveguide X-band Doppler transceivers, for example, have been shown to detect respiratory rate and heart rate of a relatively still and isolated subject (e.g., low noise environments from background scatter). Further, Doppler sensing with communications signals in the 800-2400 MHz range has been demonstrated for both detection of surface and internal heart and respiration motion, and higher frequency signals e.g., in the 10 GHz range, have been demonstrated for detection of cardiopulmonary motion at the chest surface, even through clothing. While reliable heart and respiration rate extraction can be performed for relatively still and isolated subjects, it is a major challenge to obtain useful data in the presence of random motion of the human target, peripheral human subjects, other moving objects, unknown or known number of subjects with range, and so on.

Many contact (such as ECG, EEG) and non-contact medical measurements (such as fMRI) also suffer from motion artifacts due to random motion of the subject during measurements. Various Digital Signal Processing (DSP) techniques have been used to extract useful data from such measurements. When Doppler radar sensing is performed at a close proximity with the subject (e.g., less than 1 meter), similar motion artifacts from a subject's random motion are encountered, and can be filtered out from the signal; however, if Doppler radar sensing is performed at a distance (e.g., greater than 1 meter), motion in the subject's background from other subjects and objects, in addition to movements by the subject's hands, head, etc. may affect the measurement. The use of higher (millimeter-wave) frequencies and more directive antennas may help avoid some background motion and noise; however, such systems are generally costly, require accurate aiming at the subject, and allow monitoring of only one subject at the time.

Accordingly, background noise (including both environment noise and the presence of multiple subjects) has been a barrier to many aspects of Doppler sensing of physiological motion such as cardiopulmonary information, whether from a single subject or multiple subjects.

### BRIEF SUMMARY

According to one aspect of the present invention a system and method are provided for determining presence and/or physiological motion of at least one subject using a Doppler radar system. In one example, the apparatus includes at least two inputs for receiving a transmitted signal (e.g., a continuous wave signal), the transmitted signal modulated during reflection from at least one subject, and logic (e.g., hardware, software, and/or firmware; digital and/or analog logic) for determining presence and/or physiological motion associated with the at least one subject (e.g., a heart rate and/or respiration rate of the subject). In one example the logic includes comparing (e.g., mixing) the received signal with the source signal. The apparatus may further comprise logic for quadrature detection of the received signals, and may include various blind source separation algorithms for detecting signals associated with separate subjects.

The apparatus may further include one or more transmitter antennas for transmitting the source signal. The apparatus may further comprise or access logic for encoding signals for transmission via the antennas, and in one example, vector encoding logic for causing transmission of orthogonal signals via at least two antennas.

In another example, apparatus for determining presence and/or physiological motion of multiple subjects includes a transmitter antenna for transmitting a source signal, at least two receiver antennas for receiving the transmitted signal, and logic for comparing the received signal with the transmitted signal for determining a number of subjects modulating the signal. The comparison of the signals may indicate how many subjects are within range of the transmitted signal, e.g., and have reflected the transmitted signal. The apparatus may further include logic for isolating at least one subject and/or determining cardiopulmonary motion associated with at least one subject.

The apparatus may further comprise multiple antennas, and may comprise or access logic for encoding signals for transmission via the multiple antennas. The apparatus may further comprise logic for quadrature detection of the received signals, and may include various blind separation algorithms for detecting signals associated with separate subjects.

In another aspect and example of the present invention, subjects may include or wear a transponder that moves with the motions of the body and works with incident Doppler radar signals to produce a return signal that may be more readily detected and/or isolated; for example, altering the transmitted signal in frequency and/or time may allow for improved isolation of received signals associated with subjects from noise and/or extraneous reflections. The transponders may additional detect and encode biometric information. Additionally, such transponders may assist in distinguishing detected subjects from other subjects (e.g., subject A from subject B, doctor from patient, rescuer from injured, and so on), whether or not the other subjects are also wearing transponders.

According to another aspect of the present invention, a method for determining presence and/or physiological motion of multiple subjects is provided. In one example, the method includes receiving a signal at two or more receivers, the signal associated with at least one source signal and modulated by motion of a plurality of subjects. The method further including comparing the received signals with the at least one source signal and determining a number of subjects modulating the source signal. The method further includes isolating at least one subject and determining cardiopulmonary motion associated therewith.

According to another aspect of the present invention, a computer program product comprising computer program code for determining presence and/or physiological motion of multiple subjects is provided. In one example, the product comprises program code for determining physiological motion associated with at least one subject based on a source signal and a received transmitted source signal. For example, the program code may analyze a mixed signal of the received signal and the source signal according to various algorithms to determine cardiopulmonary motion, isolate and track subjects, and the like.

According to another aspect of the present invention a system and method are provided for determining presence and/or physiological motion of at least one subject using a Doppler radar system having an analog or digital quadrature receiver. In one example, the apparatus includes a transmitter for transmitting a source signal, a quadrature receiver for receiving the source signal and a modulated source signal (e.g., as reflected from one or more subjects), and logic for mixing the source signal and the received modulated source signal to generate in-phase (I) and quadrature (Q) data, whereby nulls in the signal are avoided. In one example, the quadrature receiver further includes logic for center tracking for quadrature demodulation. The apparatus may further include logic for determining physiological motion (e.g., heart rate and/or respiration rate of a person) of a subject based on the source signal and the modulated source signal.

The apparatus may further include logic for arctangent demodulation of the I and Q data, and in another example, logic for removing DC offsets from the I and Q data (whether the DC components is from objects in range or components of the receiver). The apparatus may further include logic for measuring and/or compensating for phase and amplitude imbalance factors. In one example, the apparatus may include a phase shifter for introducing a local oscillator (LO) signal, and determining phase and amplitude imbalance between the received signal and the LO signal. The apparatus may further include a voltage controlled oscillator for providing both the transmitted and LO signals, wherein the LO signal is further divided to provide two orthonormal baseband signals.

According to another aspect of the present invention a data acquisition system for Doppler radar sensing of present and physiological motion is provided. In one example, the data acquisition apparatus includes an analog to digital converter, and an automatic gain control unit, where the analog to digital converter and the automatic gain control unit are configured to increase the dynamic range of the system, by modifying the DC offset value and gain for the signal of interest. Additionally, the system may include a first analog to digital converter and a DAC for acquiring a DC offset value and outputting a reference, as well a VGA and a second analog to digital converter for providing feedback for the automatic gain control unit. The data acquisition system may further include logic for performing arctangent demodulation of the received signals.

According to another aspect and example, a method for determining presence and/or physiological motion of at least one subject using a quadrature Doppler receiver is provided. In one example, the method comprises receiving a source signal and a modulated source signal, the modulated source signal associated with a transmitted signal reflected from at least one subject, and mixing the source signal and the modulated signal to generate in-phase (I) and quadrature (Q) data. The method may further include various demodulation methods, e.g., linear and non-linear demodulation processes.

According to another aspect and example of the present invention, a computer program product comprising computer-readable program code for determining physiological presence and motion of a subject in a Doppler radar system is provided. In one example, the product comprises program code for determining physiological motion associated with at least one subject from in-phase (I) and quadrature (Q) data output from a quadrature receiver and based on a source signal and a modulated source signal having been modified by at least one subject. The program code may further include program code for various demodulation methods, e.g., linear and non-linear demodulation processes.

According to another aspect of the present invention a system and method are provided for detecting physiological motion of at least one subject using a Doppler radar system and determining a number of subjects within range. In one example, the apparatus includes a receiver for receiving a transmitted source signal, the transmitted source signal modulated by at least one subject, logic for mixing the received transmitted signal and a local oscillator signal, and logic for performing a Generalized Likelihood Ratio Test (GLRT) on the mixed signal to determine a number of subjects modulating the signal.

According to another aspect, a method for determining a number of subjects in Doppler radar system is provided. In one example, the method includes receiving a transmitted source signal, the transmitted source signal modulated by at least one subject, mixing the received transmitted signal and a local oscillator signal, and performing a generalized likelihood ratio test on the mixed signal to determine a number of subjects modulating the signal.

According to another aspect, a computer program product comprising computer-readable program code for determining a number of subjects in a Doppler radar system is provided. In one example, the program code is for performing a generalized likelihood ratio test on a mixed signal of a received transmitted signal modulated by at least one subject and a source signal, and determining a number of subjects modulating the received transmitted signal.

The various aspects and examples of the present inventions are better understood upon consideration of the detailed description below in conjunction with the accompanying drawings and claims.

### BRIEF DESCRIPTION OF THE DRAWINGS

FIG. 1 illustrates an exemplary system for sensing physiological movement of a subject.

FIG. 2A illustrates an exemplary system for sensing physiological movement of a subject using a quadrature receiver.

FIG. 2B illustrates an exemplary system for sensing physiological movement of a subject using multiple quadrature receivers.

FIG. 2C illustrate another exemplary Doppler radar system architecture.

FIG. 3 illustrates an exemplary method for sensing physiological motion associated with a subject.

FIG. 4 illustrates a block diagram of an exemplary Single Input Multiple Output (SIMO) system for detection of physiological motion and/or the number of subjects.

FIG. 5 illustrates an exemplary Multiple Input Multiple Output (MIMO) system for detection of physiological motion and/or the number of subjects.

FIG. 6 illustrates an exemplary method for sensing physiological motion associated with a subject with a SIMO or MIMO system

FIG. 7 illustrates an exemplary multistatic Doppler radar system which may be use with a SIMO or MIMO system.

FIGS. 8A and 8B illustrate exemplary monostatic and multistatic architectures.

FIG. 9 illustrates a plot of a pre-envelope reference ECG signal from a heartbeat signal measured using a finger pulse sensor.

FIG. 10 illustrates a comparison of various exemplary algorithms; specifically, failure rate as a function of SNR.

FIG. 11 illustrates an exemplary Doppler sensing system and an exemplary transponder tag, which may be wearable by a subject.

FIGS. 12A and 12B illustrate another exemplary Doppler sensing system and an exemplary transponder tag.

FIG. 13 illustrates an exemplary thermally-variable RF inductor for use with a transponder.

FIGS. 14A-14G illustrate an exemplary fabrication process for fabricating a transponder.

FIGS. 15 and 16 illustrate exemplary performance data of different demodulation methods.

FIG. 17 illustrates an exemplary system for measuring imbalance factors of an exemplary quadrature receiver.

FIG. 18 illustrates data for an exemplary phase shifter control voltage system.

FIG. 19 illustrates an exemplary Doppler radar system according to another example.

FIGS. 20A-20C illustrate exemplary data according to illustrative examples.

FIGS. 21A and 21B illustrate exemplary systems for DC offset measurements.

FIGS. 22-25 illustrate exemplary arctangent demodulated signal data.

FIGS. 26 and 27 illustrate data associated with exemplary center tracking methods and systems.

FIG. 28 illustrates an exemplary data acquisition system.

FIGS. 29-31 illustrate exemplary data according to GLRT methods.

FIG. 32 illustrates exemplary data according to one example of detecting cardiopulmonary movement of a subject.

FIG. 33 illustrates exemplary data for the separation of two heartbeats using a CM algorithm.

### DETAILED DESCRIPTION

The following description is presented to enable a person of ordinary skill in the art to make and use various aspects of the inventions. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the inventions. Thus, the present inventions are not intended to be limited to the examples described herein and shown, but are to be accorded the scope consistent with the claims.

The following description begins with a broad description of various exemplary Doppler radar sensing systems and methods, which may be used to detect the presence of subjects through barriers (e.g., through clothing and walls) and detect presence and monitor physiological motions such as a subject's heart beat and respiration rate. This is followed by exemplary devices, algorithms, and methods, which may be utilized (alone or in combination) with the various exemplary Doppler radar sensing systems and methods to determine the number of subjects within range of a system, separate and isolate subject's motion data from noise as well as other subjects, and the like.

Exemplary Doppler Radar Sensing Systems and Methods

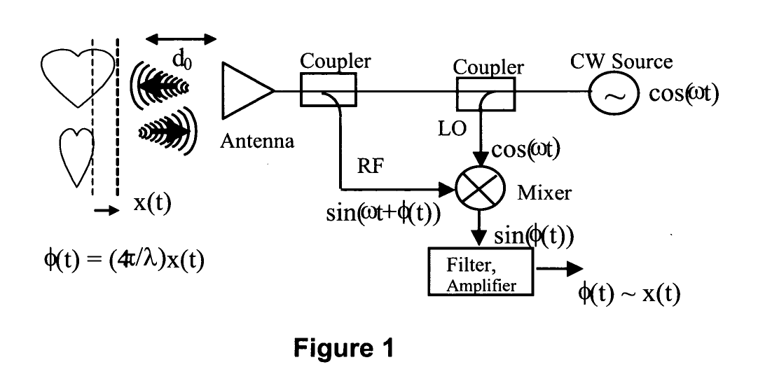

FIG. 1 illustrates an exemplary Doppler radar system having a single input single output antenna for measuring physiological motion (e.g., chest motion) associated with respiration and/or heart activity. The exemplary Doppler radar system comprises a continuous wave (CW) radar system that transmits a single tone signal at frequency f. The transmitted signal is modulated upon reflection from a subject at a nominal distance dₒ, with a time-varying displacement given by x(t). For example, the reflected signal may be amplitude, frequency, and phase modulated. Assuming small periodic displacement of the subject, for example, due to respiration and/or heart activity, phase modulation may be determined from the received signals (note that internal body reflections are greatly attenuated, more severely with increasing frequency, and can generally be dismissed depending on the particular frequency and system). At the receiver, neglecting residual phase noise, the received signal can be given by R(t) in Equation 1, where λ is the wavelength of the CW signal:

$$R ( t ) \approx cos [ 2 π ft - \frac{4 π {d}_{o}}{λ} - \frac{4 π x ( t )}{λ} ] \qquad ( 1 )$$

The received modulated signal is related to the transmitted source signal with a time delay determined by the nominal distance of the subject, and with its phase modulated by the periodic motion of the subject. The information about the periodic subject motion can be extracted if this signal is multiplied by a local oscillator (LO) signal that is associated with the transmitted source signal as illustrated in FIG. 1. For example, when the received and LO signals are mixed and then low-pass filtered, the resulting baseband signal contains the constant phase shift dependent on the distance to the subject, dₒ, and the periodic phase shift resulting from subject motion.

If the received signal and the LO signal are in quadrature, and for displacement small compared to the signal wavelength, the baseband output is approximately proportional to the time-varying periodic chest displacement, x(t). The amplitude of the chest motion due to respiration is expected to be on the order of 10 mm, and due to heart activity on the order of 0.1 mm. Even though the exact shape of the heart signal depends on the location of the observed area on the subject, overall characteristics and frequency content are generally similar throughout the chest. Since microwave Doppler radar is expected to illuminate a whole chest at once, the detected motion will be an average of local displacements associated with particular chest areas.

Although illustrated as a CW radar system, other Doppler radar systems are possible. For example, a frequency modulated CW (FM-CW) radar system or a coherent pulsed radar system may be similarly constructed and used for detecting physiological motion of a subject. Additionally, exemplary radar system described here transmit a source signal having a frequency in the range of 800 MHz to 10 GHz, but lower or higher frequencies are contemplated and possible.

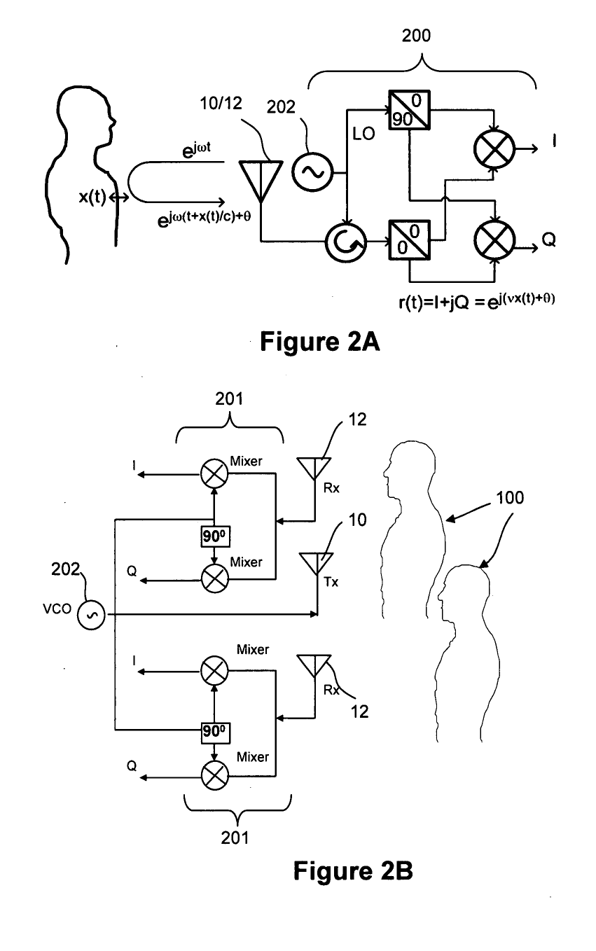

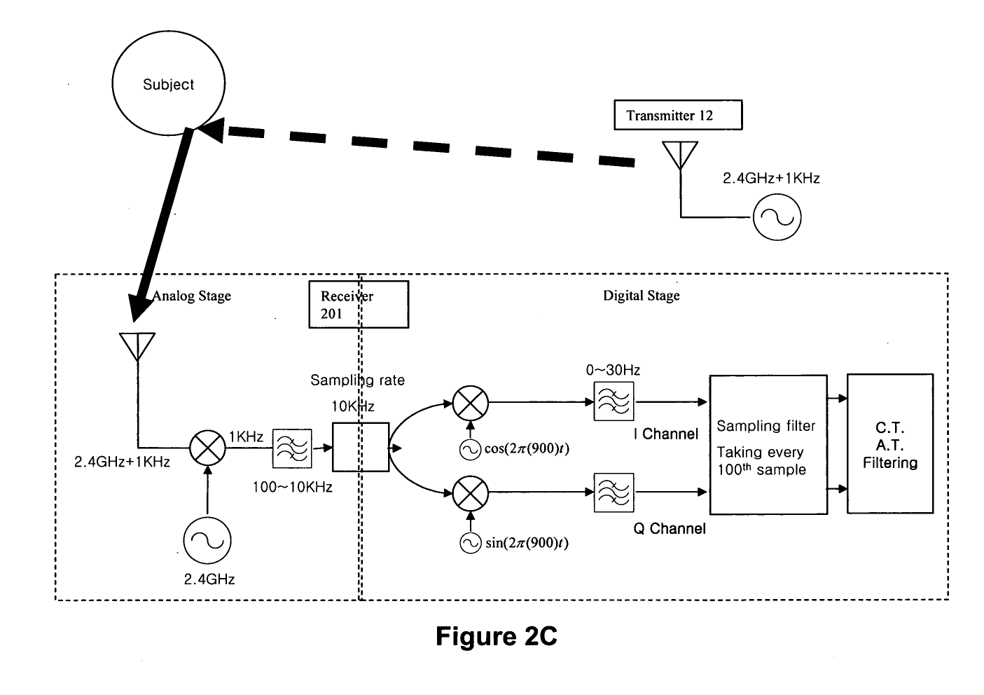

Other exemplary transmitter transceiver systems for determining presence and/or physiological motion are illustrated in FIGS. 2A-2C. With reference to FIG. 2A, a direct-conversion Doppler radar system with a quadrature receiver 200 and transmitter/receiver antenna 10/12 is illustrated. The exemplary system operates to extract the phase shift proportional to physiological displacement, e.g., due to cardiopulmonary activity. In particular, a voltage controlled oscillator (VCO) 202 provides both the source signal for transmission and a local oscillator (LO) signal. The LO signal is divided by a two-way 90° splitter to obtain two orthonormal baseband signals for mixing with the received, modulated signal. The two baseband signals are mixed with the received signals to provide I and Q outputs, which can be easily compared to determine phase and amplitude imbalance factors.

FIG. 2B is similar to that of FIG. 2A; however, the exemplary Doppler radar system illustrated includes two receivers 201 in communication with two receiver antennas 12 and at least one transmitter antenna 10 (which could be shared with one of the receiver antennas similar to that of FIG. 2A). In other examples, transmitter antenna 10 may be located remotely to one or both of receivers 201 and receiver antennas 12 (for example, with a separate device). In this example, both receivers 201 are quadrature receivers, receiving the transmitted source signal from the VCO and mixing appropriately with the received signals, including splitting the source signal with 90° splitters and mixing with the received transmitted signal (which is modulated due to reflection from one or more subjects 100). As described in greater detail below, multiple receivers may allow for detection of multiple subjects, location of the subjects, isolation of subjects, and so on. It is further noted that an antenna may operate as both a transmitter and receiver of the signal (e.g., as shown in FIGS. 1 and 2A).

FIG. 2C illustrate another exemplary digital IF Doppler radar system architecture. In this example, the receiver 201 includes an analog and digital stage as shown. Additionally, exemplary components and component values are shown; however, it will be understood by those of ordinary skill in the art that a digital system may be implemented in various other fashions. Further, the transmitter 12 may be remote to or local to the receiver 201, and receiver 201 may be implemented with a SIMO or MIMO system.

It will be recognized by those of ordinary skill in the art that various other components and configurations of components are possible to achieve the described operation of the receivers. Further, various Doppler radar sensing systems and methods described herein may be implemented alone or in combinations with various other system and methods. For example, a system may combine exemplary systems described with respect to FIGS. 1, 2A-2C to include one or more transmitters and one or more receivers (and associated antennas).

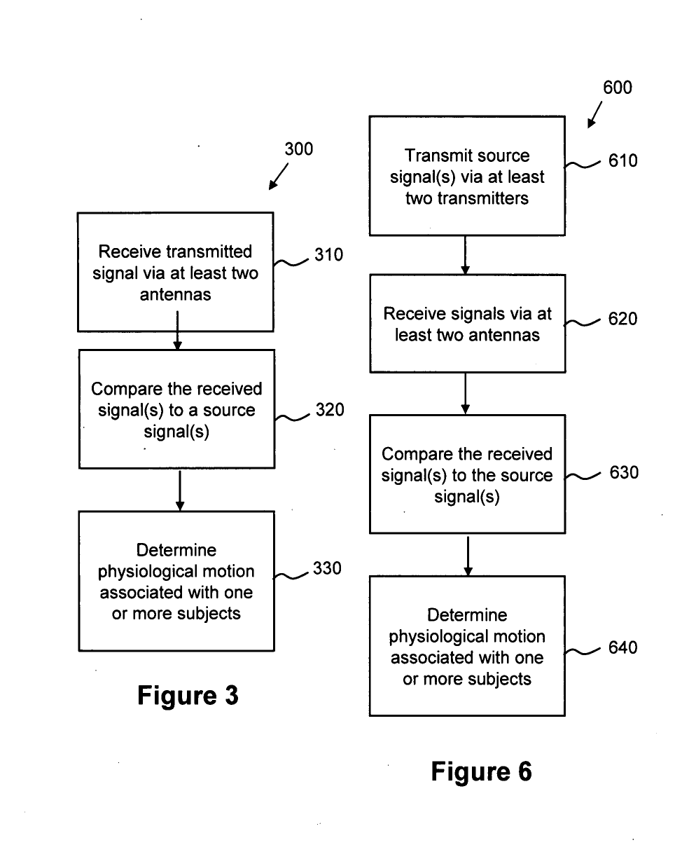

FIG. 3 illustrates an exemplary method 300 for determining presence and physiological motion of at least one subject using a multiple antenna system such as that illustrated in FIG. 2B or 2C. The exemplary method includes receiving a transmitted signal via at least two antennas, each in communication with a receiver at 310. The received transmitted signal having been modulated due to reflection from physiological motion of at least one subject. In one example, each receiver includes a quadrature receiver for mixing the received modulated signal with a source signal at 320. For example, an LO signal associated with the source signal transmitted (whether transmitted locally or remotely to the receivers) is mixed with the received modulated signal. The method further includes determining a characteristic of physiological motion associated with a subject at 330 based, at least in part, on the comparison of the received transmitted signal (having been modulated by a subject) and the source signal.

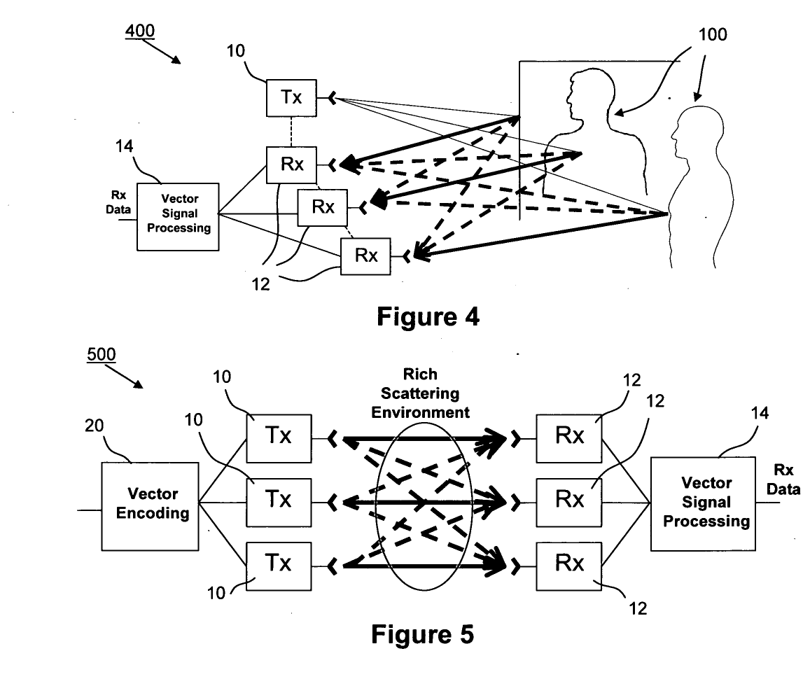

FIGS. 4 and 5 illustrate an exemplary single input multiple output (SIMO) system and an exemplary multiple input multiple output (MIMO) system respectively. SIMO and MIMO architectures, similar to those illustrated in FIGS. 4 and 5, have been used in wireless communication systems, e.g., to provide diversity gain and enhance channel capacity respectively.

A MIMO architecture as employed for a wireless communication system, for instance, takes advantage of random scattering of radio signals between transmitter and receiver antennas. This scattering is conventionally known as multipath, since it results in multiple copies of the transmitted signal arriving at the receivers via different scattered paths. In conventional wireless systems, however, multipath may result in destructive interference, and is thus generally considered undesirable. However, MIMO systems may exploit multipath to enhance transmission accuracy by treating scattering paths as separate parallel subchannels. One known technique includes Bell-Labs Layered Space-Time (BLAST), which is described, e.g., in “Layered Space-Time Architecture for Wireless Communication in a Fading Environment When Using Multiple Antennas,” Bell Labs Technical Journal, Vol. 1, No. 2, Autumn 1996, pp. 41-59, and which is incorporated herein by reference. Broadly speaking, the BLAST technique includes splitting a user's communication data stream into multiple substreams, using orthogonal codes in the same frequency band, where each transmitter antenna transmits one such substream. On the other end, each receiver antenna receives a linear combination of all transmitted substreams, and due to multipath, these combinations are slightly different at each receiver antenna.

While SIMO systems in wireless communications can provide diversity gain, array gain, and interference canceling gain, they provide only one source signal. In the case of Doppler radar, however, for a single transmitter antenna, there are essentially as many independent signals as there are scatterers because a subject and objects in the subject's vicinity will scatter signal waves (thereby acting as secondary sources) resulting in independent phase shifts as illustrated in FIG. 4. For example, with the use of N receiver antennas, N linear combinations of scattered signals are received. Additionally, each transmitter Tx antenna in a Doppler radar system can be identified by orthogonal codes or slightly shifted frequencies to simplify channel estimation.

With reference to FIG. 4, various exemplary algorithms and hardware implementations for SIMO Doppler sensing of presence and physiological movement (e.g., cardiopulmonary activity) of one or more subjects are now described. In particular, exemplary SIMO system 400 includes a transmitter (Tx) 10, for transmitting a signal and multiple receivers (Rx) 12 (e.g., at least two receivers 12). Additionally, SIMO system 400 includes vector signal processing apparatus 14, comprising logic for analyzing received signals according to the various examples provided herein. For example, vector signal processing apparatus 14 may comprise logic operable for receiving signals associated with the received modulated signals (e.g., from one or more subjects 100) and determining physiological movement associated of with one or more subjects. Specifically, vector signal processing apparatus 14 includes logic (e.g., hardware, software, and/or firmware) operable to carry out the various methods, processes, and algorithms described herein. For instance, the logic may be operable to demodulate received signals, perform Blind Source Separation (BSS) processes (of which exemplary BSS methods are described in greater detail below), determine the number of subjects modulating the received signals, determine heart rate and/or respiration rate of one or more subjects, and so on as described herein. Additionally, it should be understood that vector signal processing apparatus 14 may be in communication with transmitter 10 or vector encoding apparatus 20 (e.g., to receive the source signal itself or other data associated with the transmitted signal).

In one example, receivers 12 are configured as quadrature receivers (e.g., as described with reference to FIGS. 2A-2C). Initially, to describe the exemplary operation of SIMO system 400 according to one example, a simple case of a signal received from a single subject 100 arriving through a single path at receivers 12 is considered. In such an instance, the sampled, baseband received signal at the n-th receiver 12 in SIMO system 400 with quadrature receivers can be written as

$${r}_{n} ( t ) = exp ( j ( Kx ( t ) + n ( ϕ ) ) + {w}_{n} ( t ) , \\ n = 0 … M - 1 \\ ϕ = \frac{2 π d}{λ} sin ( v ) , K = \frac{4 π}{λ}$$

for a linear array, with angle of incidence v and noise wₙ(t). If the signal received at the M receivers is collected into a vector, this can be written as
r(t)=exp(j(Kx(t))s(φ)+w(t)
s(φ)=[1,exp(jφ), . . . , exp(j(M−1)φ)]^{T}

If the signal arrives through several paths with different angle of incidences (e.g., as illustrated in FIG. 4 due to various objects and subjects in the environment), the received signals can be written as

$$\begin{matrix}r ( t ) = {\sum }_{p = 1}^{P} exp ( j ( Kx ( t ) ) s ( {ϕ}_{p} ) + w ( t ) \\ = exp ( j ( Kx ( t ) ) s + w ( t )\end{matrix}$$

Thus, the received signals may be characterized by a characteristic vector s. If there are S subjects 100 at different locations, as illustrated in FIG. 4, they will likely have different direction of arrival (DOA) vectors s, and the total received signal can be written as

$$\begin{matrix}r ( t ) = {\sum }_{s = 1}^{S} exp ( j ( {Kx}_{s} ( t ) ) {s}_{s} + w ( t ) \\ = Mx ( t ) + w ( t )\end{matrix} \\ M = [ {s}_{1} , {s}_{2} , … {s}_{S} ] \\ x ( t ) = {[ exp ( j {Kx}_{1} ( t ) ) , exp ( j {Kx}_{2} ( t ) ) , … , exp ( j {Kx}_{S} ( t ) ) ]}^{T} \qquad ( 2 )$$

The resulting matrix is an M×S matrix. If subjects 100 are moving (e.g., the subject is changing positions as opposed to periodic motion associated with heart rate and respiration), M will additionally be a time-varying matrix; however, a stationary example will be described first. Two exemplary methods are provided for the above M×S matrix; a disjoint spatial-frequency method and a joint spatial-frequency method.

Initially, it should be recognized that the problem stated in equation (2) can be considered a blind source separation (BSS) problem, in which case each signal in x(t) is modeled as a random signal and a suitable BSS algorithm may be applied for determining the number of subjects and physiological motion thereof.

The method further includes determining the number of subjects using BSS. In one example, this is done by first separating sources using a BSS algorithm tailored to extracting respiration and heartbeat, as opposed to a general BSS algorithm (of which an example is described below). The separated sources are then examined to determine if they are actual sources of physiological motion, for example by a GLRT algorithm (described herein) or the like.

The method may further comprise separating the heart and respiration signals and tracking the heart rate and respiration rate. In one example, the method includes separating the heart and respiration rates in the frequency domain (e.g., via suitable filtering techniques). More advanced approaches, such as adaptive filtering processing methods may also be used, and in one example, since the respiration signal is much stronger, one exemplary method includes determining the respiration signal using a parametric model, and then subtracting the signal, similar to interference cancellation used in conventional CDMA and ECG techniques.

A second exemplary method for the M×S matrix includes the joint spatial-frequency method. In comparison, the disjoint approach above approximates the source signals x(t) to be stationary and are separated in the spatial domain. One consequence is that the disjoint approach can typically only separate M−1 subjects. Improved performance may be achieved if the signal r(t) is examined in both space and frequency. Different sources can be expected to have both different spatial and frequency signatures resulting in a 2-dimensional source separation problem. Further, since heart and breathing rates are time-varying, exemplary time-frequency analysis methods, such as wavelet transforms, are described.

If a subject moves (e.g., in addition to cardiopulmonary motion), the effect on equation (2) is twofold. First, assuming an approximately constant motion, the effect on the received signal is a constant frequency shift, i.e., the baseband received signal will be exp(j(Kxₛ(t)+ωₘt)). Second, the mixing matrix M becomes time-varying. Conventional BSS algorithms and methods are typically used in application having stationary sources with a few exceptions, e.g., relating to speech separation such as “Dynamic Signal Mixtures and Blind Source Separation,” Proceedings of the IEEE International Conference on Acoustics, Speech, and Signal Processing, ICASSP '99, pp. 1441-1444, March 1999, which is incorporated herein by reference.

Accordingly, in this exemplary joint spatial-frequency method, subjects are isolated and tracked according to their movement. An exemplary method for tracking subjects according to their movement can be achieved through filtering, e.g., with an adaptive filter or Kalman filtering as described by S. Haykin, “Adaptive Filter Theory,” 4^{th} edition, Prentice-Hall, N.J., 2002, which is incorporated herein by reference. As the moving subjects are tracked by receivers 12 the heart rate and respiration rate data may be extracted from the received signals. In another example, subjects can be tracked, and their heart rate determined during pauses in motion (e.g., although subjects may move around in a room, they are stationary most of the time).

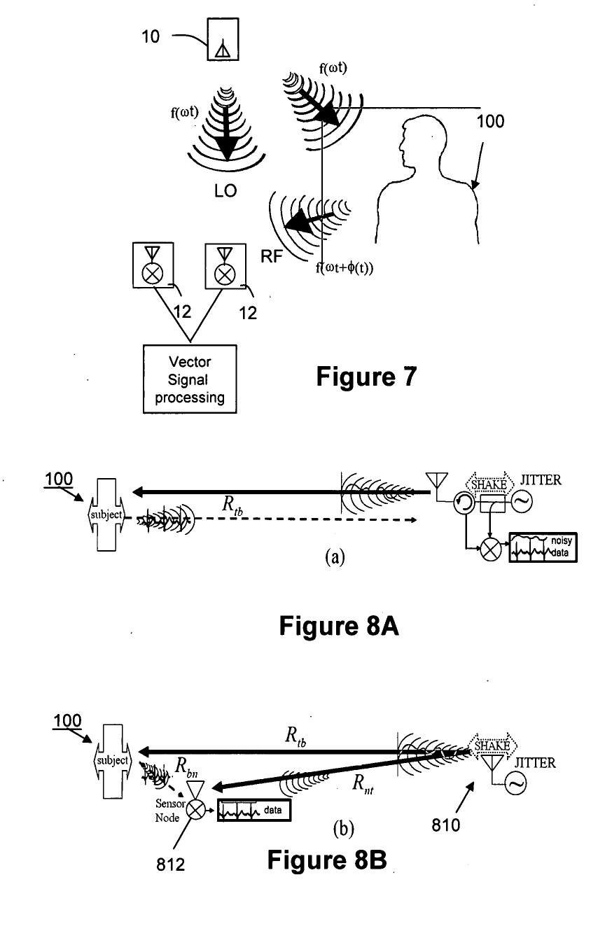

With reference again to FIG. 5, exemplary MIMO system 500 will be described in greater detail. MIMO system 500 is similar to SIMO system 400, however, multiple transmitters (Tx) 10 for transmitting multiple source signals and multiple receivers (Rx) 12 (similar to SIMO system 400, which may include quadrature receivers) are implemented. Additionally, MIMO system 500 includes vector encoding apparatus 20, comprising logic for encoding signals for transmission via transmitters 10, and vector signal processing apparatus 14, comprising logic for analyzing received signals as described. It should be noted that the components illustrated in FIG. 5 may be included with a single apparatus or system, or divided (e.g., by transceiver and receiver side) in various fashions; for example, a single chip or package (see, e.g., FIG. 7) may include both a transmitter 10 and receiver 12 associated therewith.

MIMO systems may be divided generally into non-coherent systems and coherent systems. An exemplary non-coherent MIMO system comprises N transmitters 10 with a transmitter antenna associated with each. Further, transmitters 10 may be spatially separated and may use unsynchronized oscillators. Each transmitter 10 may be controlled (e.g., via vector encoding apparatus 20) such that each transmitter 10 transmits a different modulated signal. In one example, the transmitted signals are orthogonal, which may be achieved in different ways; for example, the transmitters can transmit at different times, at different frequencies, or using different codes. These three approaches correspond generally to TDMA, FDMA, and CDMA multiple-access in communication systems. A CDMA approach could use one of a number of different designs of (near) orthogonal codes for MIMO communication systems. With orthogonal transmission, the different signals may be completely separated at the receiver using a matched filter. The received signal due to the i-th transmitter can then be written as:
rᵢ(t)=Mᵢx(t)+wᵢ(t)

This can be collected into a larger vector

$$[ \begin{matrix}{r}_{1} ( t ) \\ ⋮ \\ {r}_{N} ( t )\end{matrix} ] = [ \begin{matrix}{M}_{1} \\ ⋮ \\ {M}_{N}\end{matrix} ] x ( t ) + w ( t ) \qquad ( 3 )$$

Note that n(t) is still white Gaussian noise due to the orthogonality of the transmitted signals. Further, the total matrix is an MN×S matrix, and that all the Mᵢ matrices can be different. The system is similar to SIMO system 400 described previously, and the algorithms described there can be used in a similar fashion. Thus, an (N, M) MIMO system can allow for the separation of a number of subjects proportional to MN, whereas using M+N antennas at a receiver only allows for separation of a number of subjects proportional to M+N (assuming the total matrix has full rank, and this will in practice give the limit of the resolution).

If the transmitters are not controlled, for example, relying on existing signal sources in the environment (e.g., pseudo-passive sensing), the system may operate without explicitly separating the transmitters, operating as a SIMO system. In some examples, however, it is possible to separate the individual sources; for example, if the sources used are CDMA cell-phone signals, different cell-phones use different codes, which can be separated blindly without knowledge of the codes. Once the transmitter sources have been identified, a suitable BSS algorithm or method can be used to separate the signal sources as described above.

In an exemplary coherent MIMO system the N transmitter antennas are located with or synchronized with a single transmitter 10 (e.g., via vector encoding apparatus 20) and synchronized to the same source/LO carrier. Further, instead of letting each antenna transmit an independent signal, all antennas transmit Q orthogonal signals (where Q might be larger or smaller than N), as follows

$$h ( t ) = {\sum }_{q = 1}^{Q} {a}_{q} {h}_{q} ( t )$$

where a_{q} is a complex vector. As for the coherent system, the Q orthogonal systems can be separated at the receiver 12 by matched filtering. The received signal due to a single subject for the q-th transmitted signal is now modified to

$$\begin{matrix}{r}_{q} ( t ) = {\sum }_{n = 1}^{N} {\sum }_{p = 1}^{P} exp ( j ( Kx ( t ) ) {a}_{q , n} s ( {ϕ}_{p , n} ) + w ( t ) \\ = exp ( j ( Kx ( t ) ) s ( {a}_{q} ) + w ( t )\end{matrix}$$

and the total received signal
r_{q}(t)=M_{q}(a_{q})x(t)+w(t)
M_{q}(a_{q})=[s₁(a_{q}),s₂(a_{q}), . . . s_{S}(a_{q})] (4)

and the received signal from all q transmitted signals

$$[ \begin{matrix}{r}_{1} ( t ) \\ ⋮ \\ {r}_{Q} ( t )\end{matrix} ] = [ \begin{matrix}{M}_{1} ( {a}_{q} ) \\ ⋮ \\ {M}_{Q} ( {a}_{q} )\end{matrix} ] x ( t ) + w ( t )$$

The difference between equation (4) and (3) includes that the system (e.g., vector signal processing apparatus 14) can control the mixing matrix. This may be used, for example, to maximize rank, and further to control the singular values toward the best case of having all identical singular values. In the simplest case, with no multipath, the a_{q} can be used to beamform in the direction of subjects of interest, to separate subjects or separate different parts of the torso of a single subject. Additionally, an adaptive feedback approach may be used to optimize the coefficients a_{q}.

FIG. 6 illustrates an exemplary method 600 for sensing physiological motion of at least one subject using a MIMO system such as that illustrated in FIG. 5. The exemplary method includes transmitting one or more source signals via at least two transmitters (or at least two transmitter antennas) at 610. As described previously, each transmitter may transmit the same signal, different modulated signals, orthogonal signals, etc. Method 600 further includes receiving the transmitted signal via at least two antennas, each in communication with at least one receiver device at 620, the received signal having been reflected and modulated by movement of at least one subject. In one example, each receiver includes a quadrature receiver for mixing the received modulated signal with at least one of the transmitted source signal at 630. Method 600 further includes determining a number of subjects modulating the transmitted source signals and/or a characteristic of physiological motion associated with the subjects based, at least in part, on a comparison of the received and transmitted signals.

FIG. 7 illustrates an exemplary multistatic Doppler radar sensing system and method having an array of distributed receiver nodes (which may be used similar to exemplary MIMO or SIMO system described above). In particular, a transmitter 10, remote to multiple node receivers 12, transmits a source signal (LO), which is received directly by the antenna associated with each of receivers 12. The transmitted source signal also reflects from subject 100 and is modulated accordingly. Specifically, each receiver 12 further receives the transmitted source signal modulated upon reflection with subject 100. Receivers 12, which may include quadrature receivers, mix the source signal (LO) and modulated signal (RF) to produce a phase-demodulated output, which includes components related to the motion of one or more nearby subjects 100. The mixed signals may be communicated to vector signal processing 12 (which may be local or remote to a receiver 12) for determining heart rate and/or respiratory motion, subject location, and/or the number of subjects from the signal data.

Additionally, a distributed array of receivers 12 (e.g., as a networked array of nodes), similar to a SIMO system, may be networked together to increase resolution and/or sense multiple subjects 100. In one example, where receivers 12 are distributed over large areas, e.g., on the order of several meters to kilometers or more, the source signal may be transmitted from a high altitude relative to the receivers (e.g., via a tower or helicopter). In addition to an array of receivers 12, and array of transmitters 10 are also possible, similar to the described MIMO systems.

In one example, a multistatic architecture may further compensate for vibrations of the transceiver, e.g., from user “hand-shake,” by leveraging the array of receiver nodes. FIGS. 8A and 8B illustrate exemplary monostatic and multistatic Doppler radar sensing systems, respectively; and in particular, FIG. 8B illustrates an exemplary system and method for compensating for shake or jitter of a transmitter and/or receiver device. FIG. 8A illustrates an exemplary mono-static direct-conversion microwave Doppler radar system. Phase stability of the subject measurement system affects the accuracy of the phase demodulation. For example, it has been shown that if the transmitted signal and the local oscillator (LO) are derived from the same source, the range correlation effect greatly reduces detrimental effects of electrical phase noise of the signal source. This reduction in output signal noise is inversely proportional to the phase delay between the local oscillator and the received phase modulated signal. If the transceiver is a hand-held device, which could be for example used for search and rescue operations or sense through the wall military applications, “hand-shake” of the user (or other vibrations on the transceiver) will introduce path length change that will appear as phase noise in the demodulated base-band signal. In case of the mono-static radar this noise does not appear in the LO path and thus there is no benefit of range correlation. Therefore such “shaking” typically results in signal degradation that obstructs the detection of cardiopulmonary signals.

The use of a bistatic or multistatic radar system with a receiver (sensor node) placed in the vicinity of the subject as illustrated in FIG. 8B may reduce the described signal degradation. In one example, receiver or node 812 comprises an antenna and a mixer operable to receive both the direct signal (LO) from the transmitter 810, and the signal reflected from subject 100. The two signals are both subject to the same “mechanical” phase noise from transmitter 810. If these path lengths are similar, there can be significant phase noise reduction due to the range correlation effect, thus enabling accurate detection subject motion.

In one example, the transmitted signal from an exemplary CW radar system has the form
Sₜ(t)=cos(ω₀t) (5)

Where ω₀ is the radian oscillation frequency. This signal reflected from the subject 100 will be demodulated at the mono-static end as

$${S}_{r} ( t ) = A cos ( - \frac{4 π {R}_{tb}}{λ} ) \qquad ( 6 )$$

where λ is the wavelength and R_{tb} is the time-varying distance of the subject's chest from the transmitting antenna. On the other hand, the total RF signal received at the multistatic sensor node 812 of FIG. 8B is

$${S}_{nRF} ( t ) = B cos ( {ω}_{0} t - \frac{{ω}_{0}}{c} {R}_{nt} ) + C cos ( {ω}_{0} t - \frac{{ω}_{0}}{c} {R}_{tb} - \frac{{ω}_{0}}{c} {R}_{bn} ) \qquad ( 7 )$$

where R_{tb} is the time-varying distance of transmitter to the subject and R_{bn} is the time-varying distance of the subject to the node. If we neglect amplitude variation due to propagation loss, mixing s_{nRF}(t) by itself by passing it through a non-linear device, results in the following base-band component

$${S}_{n} ( t ) = BC cos ( \frac{2 π}{λ} ( {R}_{tb} + {R}_{bn} - {R}_{nt} ) ) \qquad ( 8 )$$

If the mono-static antenna is located at a large distance from both the human subject and the node, such that R_{tb}≈Rₙₜ, slight physical movements of the mono-static antenna have the same effect on R_{tb} and Rₙₜ, so that they cancel each other out

$${S}_{n} ( t ) \approx BC cos ( \frac{2 π}{λ} {R}_{bn} ) \qquad ( 9 )$$

Considering equation (6) and equation (9), it will be recognized that, compared to the mono-static radar system, the received signal at the sensor node 812 is less sensitive to the R_{tb}(t), which is partly given rise to by unwanted movements of the mono-static antenna. This effect is similar to the range correlation effect which reduces the base-band noise caused by the LO's phase noise. The two signals arriving at the sensor node contain nearly the same phase variation caused by unwanted movements of the mono-static antenna. The closer the node and the subject are, the better these two phase variations cancel out resulting in a less noisy base-band signal providing more accurate life signs detection.

In another example, the effect of “handshake” may be compensated or overcome via an algorithm such as a Blind Source Separation (BSS) algorithm. Such an example will be described below under.

Blind Source Separation (BSS) Systems and Methods

According to one aspect of the invention, a Doppler radar system and method are operable to detect a number of subjects in the range of the system and separate out for detection individual signals modulated from each of the subjects. In one illustrative example, the separation (and detection) of heart rates and respiration rates of two or more subjects is achieved by the use of a Blind Source Separation (BSS) algorithm. Exemplary BSS algorithms which may be employed include a Constant Modulus (CM) algorithm, the Analytic Constant Modulus Algorithm (ACMA), the Real Analytical Constant Modulus Algorithm (RACMA), or an Independent Component Analysis (ICA) algorithm. ACMA is described in greater detail, e.g., by “An Analytical Constant Modulus Algorithm”, IEEE Trans. On Signal Processing, vol. 44, no. 5, May 1996, RACMA is described in greater detail, e.g., by “Analytical Method for Blind Binary Signal Separation,” IEEE Trans. On Signal Processing, vol. 45, Issue 4, April 1997, pp. 1078-1082; and ICA is described in greater detail, e.g., in “Independent Component Analysis, a new concept?” Signal Processing, Special issue on Higher-Order Statistics, vol. 36, no. 3, pp. 287-314, April 1994, both of which are incorporated herein by reference.

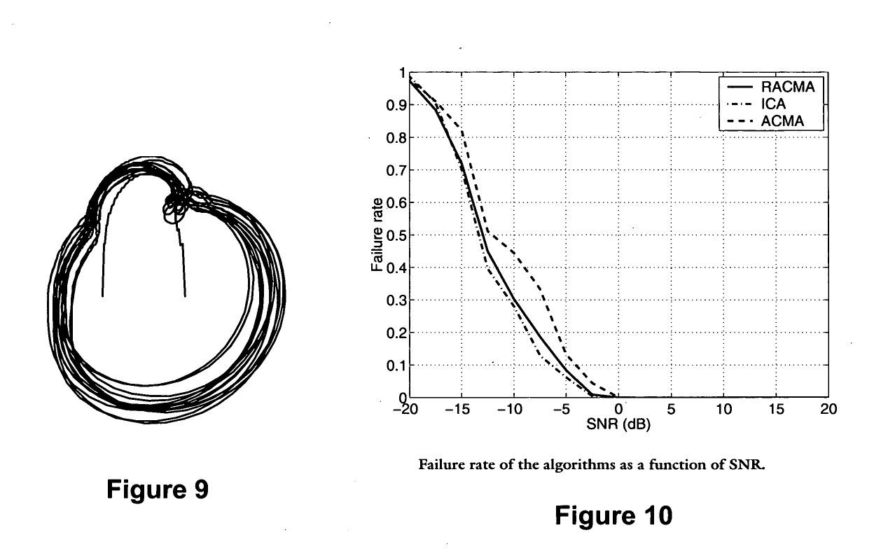

A typical heartbeat signal is not perfectly modeled by a periodic signal due to heart rate variability. Therefore, in one example, a model for the heart rate after low-pass filtering to remove harmonics may be written as:
s(t)=c(t)cos(ωₒt+φ(t)) (10)

where c(t) is a real scalar and φ(t) is a phase component that can be modeled as a random walk on the unit circle. Generally, φ(t) varies relatively rapidly and the signal cannot accurately be considered periodic; however, c(t) is nearly constant such that s(t) can be viewed as a constant modulus signal. FIG. 9 illustrates a plot of a pre-envelope reference heartbeat signal measured using a finger pulse sensor after bandpass filtering the range of 0.03-30 Hz. The pre-envelope is obtained by taking the signal and adding in quadrature its Hilbert transform. The plot is almost circular indicating that the heartbeat signals have a nearly constant modulus envelope (after low-pass filtering, this property shows up even stronger).

Accordingly, when multiple subjects are present within range of a Doppler radar system including multiple receivers (e.g., as illustrated in FIGS. 2B, 2C, 4, and 5), exemplary BSS methods described herein may be used for determining the number of sources and heartbeat/respiration rates of each of the unknown number of sources from the received mixture of signals acquired. (The term “blind” here is appropriate because only an a-priori knowledge for the signals is their statistical independence, where no other information about the signal distortion on the transfer paths from the sources to the sensors is available beforehand.

As an illustrative example, consider an M-element antenna array system and a CW radar system (e.g., as described with reference to FIG. 2B or 2C) transmitting a single tone signal at frequency c. The model (2) describes a linear mixing of the sources, and BSS methods can therefore be applied to separate and monitor sources. In (2) the source signal is exp(jKxₛ(t)), where xₛ(t) is the heartbeat and respiration signal. If the wavelength λ is large compared to the maximum displacement of xₛ(t) (which is the case at frequencies below approximately 10 GHz), the complex exponential can be approximated by
exp(jKxₛ(t))≈(1+jKxₛ(t))

It will be noted that here xₛ(t) appears as a real signal (multiplied by a complex constant). The DC offset can be ignored. A real BSS algorithm therefore should be applied. One exemplary method includes applying RACMA; in another exemplary method, a Hilbert transform is applied to the output of the antennas and calculate the analytic signal, and then a complex BSS algorithm such as ACMA is applied.

An illustrative comparison of exemplary BSS algorithms is described with reference to FIG. 10. In this particular example, ACMA, RACMA, and ICA algorithms were applied to separate two different heartbeats. In a first example, reference heartbeat signals that were recorded using finger pulse sensors and bandpass filtered were analyzed. Two reference signals from different people were assumed to pass through a typical wireless environment scenario characterized by a matrix M as in (2), and white Gaussian noise added. Simulations were conducted for scenario mimicking a 2-element receiving antenna array radar system. Further, different M matrices of Signal-to-Noise Ratio (SNR) in the semi-synthesized cases were used.

A database of heartbeat signals from finger pulse sensors was mixed pair wise to assess exemplary BSS algorithms. In particular, heartbeat signals of 10 subjects were obtained to form 45 couples. Each measurement was 700 samples long at a frequency of 20 HZ, so 35 seconds (approximately 30 beats). For each couple, the experiment was repeated 5 times with different noise, resulting in 225 independent runs.

To isolate the fundamental tone of the heartbeats, the mixed data is filtered with a band-pass filter over the range [0.75; 2] Hz. Exemplary ICA and RACMA are applied directly on the mixed data, and for ACMA the data is passed though a Hilbert transform prior to application.

The influence of the Noise power over the algorithms, the SNR ranges in [−20, . . . , 20] dB were compared. In this example, a mixing matrix was chosen to be: [1 1 1 1; 1 −1 1 −1]^{T}, which has a conditioning number of 1. Once the separation algorithm has delivered output signals, they are used to estimate the heart rate. FIG. 10 illustrates the failure rate as a function of the SNR for one particular example. As seen, the three BSS algorithms described have similar performance in this instance, with the ICA showing slighter better performance than the Constant Modulus algorithms.

In one illustrative example, which may use the exemplary transmitter-receiver system illustrated in FIG. 2B or 2C, various exemplary BSS algorithms may be used to separate subjects and detect heartbeats thereof. In this particular example, displacement due to breathing is used initially to separate the subjects, and then the same or similar beam forming vector is used to separate out the heartbeats (if the mixing matrix for respiration and heartbeat are similar). In some instances, however, subjects may not be breathing due to medical reasons or to hide; also, the respiration signal is generally more irregular, and therefore more difficult to distinguish from other movement. Accordingly, separation of subjects based on both respiration and separation of heartbeat may be used.

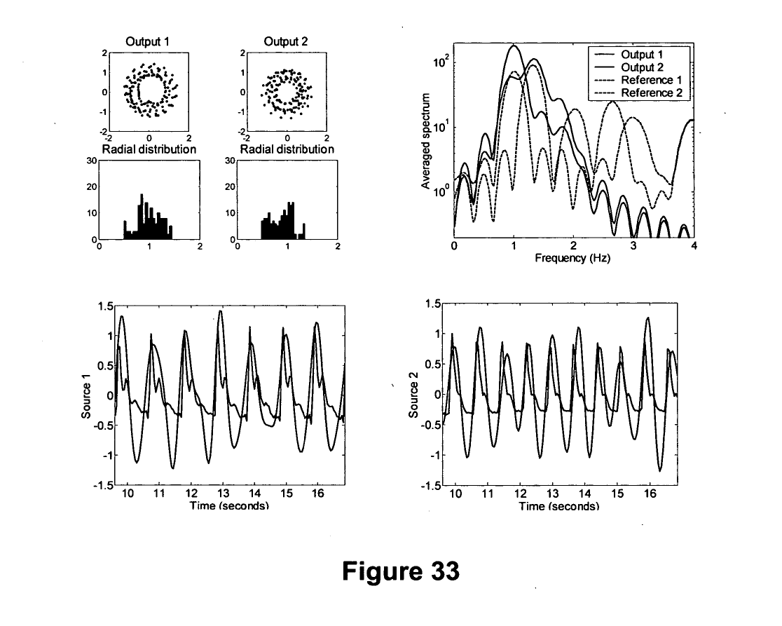

Exemplary data for the separation of two heartbeats using a CM algorithm as described herein from measured wireless data is provided in FIG. 33. The first subfigure thereof illustrates the Hilbert transform of the separated sources, verifying that they have CM property. The second subfigure illustrates the two separated sources in the frequency domain, compared with a reference signal obtained from a finger pulse monitor. The last two figures show the two separated heartbeats in the time-domain, compared with the references.

In another example of BSS, the effect of “handshake” on a received signal may be compensated or overcome. In particular, a BSS algorithm may be applied to a received signal to compensate for unwanted vibrations on the system. The strongest sources identified in the signal are typically reflections from walls and the like. If the system is a handheld device, for example, the source is generally not a DC source, but can be extracted via a suitable BSS algorithm and movement of the handheld device relative to the source (e.g., a wall) estimated. The movement may then be compensated for, e.g., subtracted form the received signal. Exemplary handshake removal via a BSS algorithm may be used in SIMO or MIMO systems (including exemplary multistatic systems as described previously).

Wearable Transponders

In another aspect and example of the present invention, subjects may include or wear a transponder operable to move with the subject's motion. The transponders may work with incident Doppler radar signals to produce a return signal that may be more readily detected and/or isolated; for example, altering the return signal in frequency and/or time may allow for improved isolation of signals associated with subjects from noise and/or extraneous reflections. Additionally, such transponders may assist in distinguishing detected subjects from other subjects (e.g., subject A from subject B, doctor from patient, rescuer from injured, and so on), whether or not the other subjects are also wearing transponders.

In one example, the transponder includes Radio Frequency-Identification (RF-ID) tag that isolates the incident signal from the return signal by a predictable shift in frequency. A simple form of this circuit can be based on a Schottky diode that multiplies the frequency of the incident signal. For example, an input of the diode is tuned or filtered for the incident source signal, and the output tuned or filtered for the desired harmonic generated at the diode. Thus, an exemplary RF-ID tag may operate to re-radiate an incident signal of frequency “ω”, at a new frequency, e.g., of “2ω”, which may be more easily isolated from the transmitted signal.

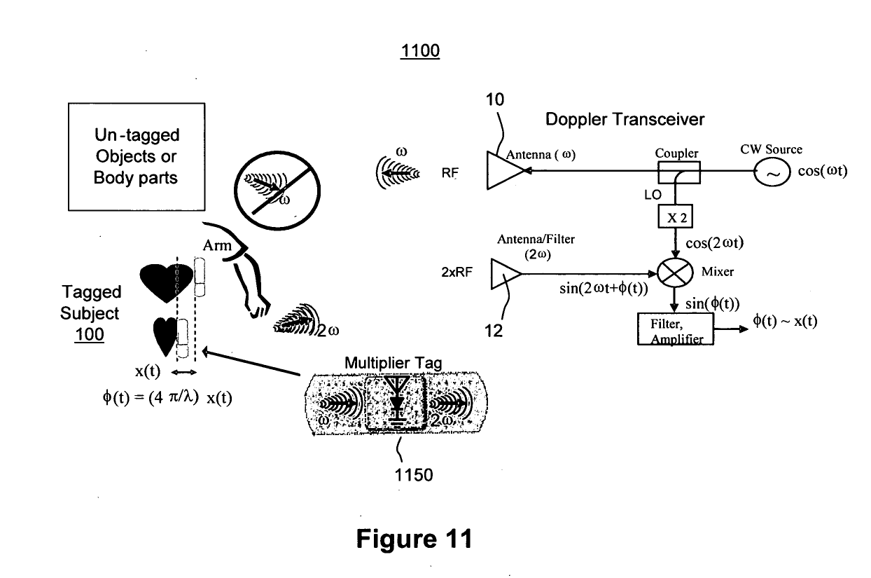

One exemplary system is illustrated in FIG. 11. Specifically, a Doppler radar sensor system 1100, which may be similar to those illustrated in FIG. 1, 2A, 2B, 2C, 4 or 5, is configured to transmit a source signal at frequency ω via antenna 10 and receive a modulated sign at frequency 2ω, via receiver antenna 12. Sensor system 1100 operates to interrogate tag 1150, in this example mounted to a chest of a subject 100 and ignore or filter return signals at the original interrogation frequency, ω, including those reflected from stationary objects, other subjects, and untagged parts of the body of the subject. In this way chest motion can be specifically detected as a Doppler phase shift in the multiplied signal only, as compared by the mixer to a correspondingly multiplied sample of the original signal.

In other examples, additional data can be introduced as modulation on the multiplying circuitry of a tag, e.g., of tag 1150. For example, electrodes adjacent the skin could be used to sense bioelectric information such as heart signals and impose such information as a bias at the diode to periodically interrupt the reflection signal. In another example, the multiplied output signal could be directed against the skin in an area where blood vessels are near the surface, and the reflected signal can be analyzed for dielectric permittivity changes associated with changing blood glucose levels.

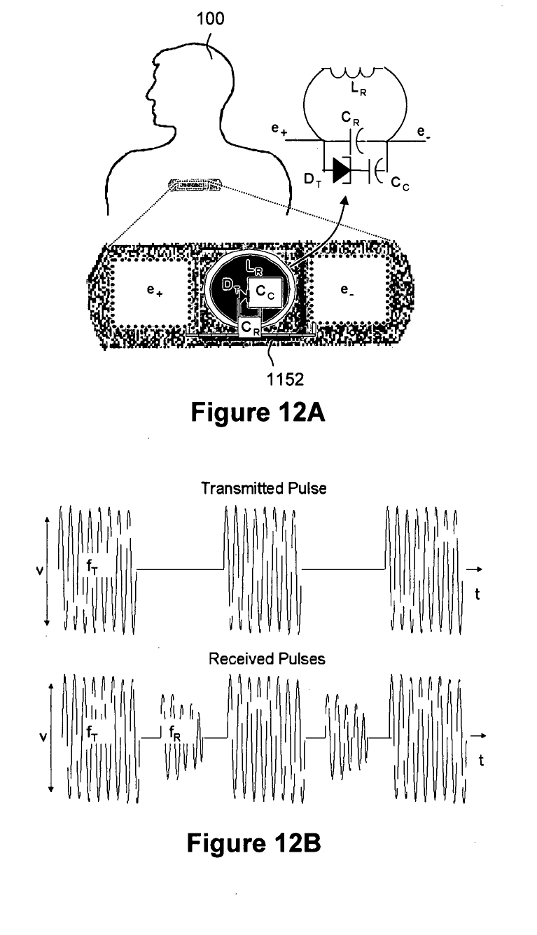

In yet other examples, suitable tag circuits can alter the return signal in time. One example includes an oscillating body-sensor, which is energized by a pulsed incident signal but re-radiates a new signal at a frequency controlled by a resonant circuit local to the tag. An exemplary tag 1152 and circuit is illustrated in FIG. 12A, and exemplary source and modulated signals are illustrated in FIG. 12B. In this example, an incident pulsed radar signal (e.g., as illustrated in FIG. 12B) couples to an inductive antenna, L_{R}, and rectification by a tunnel diode, D_{T}, charges a capacitor, C_{C}. When the incident pulse is absent, the charging capacitor discharges, and the tunnel diode oscillates at a frequency governed by the capacitor, C_{R}. The modulated signal received by a suitable receiver is illustrated in FIG. 12B.

The resonant inductive antenna or capacitor values can also be variable, and controlled or modulated by a physical parameter of interest, such as temperature, to provide additional biometric information regarding a subject. The exemplary transponder 1152 may provide the advantage of separating the incoming signal and peripheral clutter reflections from the body-scattered signal in both time and frequency, simultaneously. It will be recognized by those of ordinary skill in the art that other exemplary circuits may be used to alter the return signal in time similarly to that described here.

In addition to improving sensitivity and isolation for the Doppler return signal, transponders (such as transponder tags 1150 and 1152) can also be operable for providing additional biometric data associated with tagged subjects. For example, utilizing components in the RF circuits of a transponder that have values that are sensitive to biological parameters of interest, the reflected signal can be altered and effectively encoded with data associated with those parameters. Implementing a transponder comprising resonant inductors or capacitors with values that vary with the parameter measured, and thus affect the resonant frequency of the circuit, may be used. For instance, inductors and capacitors can be made to vary with temperature, inertia, or pressure by using these phenomena to alter the displacement between coil turns or parallel plates. Further, capacitors can further be made to vary in proportion to changes in the dielectric between plates or fingers.

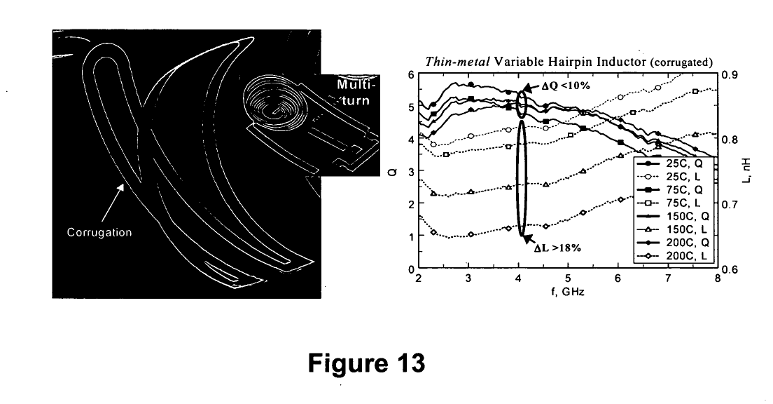

In one example, a transponder tag for providing biometric information comprises a thermally controlled variable RF inductors as illustrated in FIG. 13, which illustrates an exemplary MEMS thermally-variable RF Inductor. The inductance of the component is given by the sum of its internal and mutual (loop-to-loop) inductance. Stress between two thin-film layers (in this instance, gold and polysilicon) curves the loops proportionally to temperature; however, if the loops are designed to misalign with temperature (corrugation), a corresponding change in inductance is seen, up to 50% as illustrate in the graph to the right.

Broadly speaking, thermally controlled variable RF inductors are based on the manipulation of interlayer stress between sandwiched thin films of conductive and non-conductive material. For example, an inductor made of multiple turns that align flat in a plane at one temperature and misalign at other temperatures (with suitably designed structures) vary the mutual component of the device inductance. Such a transducer provides the necessary frequency shift in time/frequency shifting tag circuits. In other example, parallel plate capacitors can be arranged to similarly deform with temperature resulting in a change in the plate spacing, and thus the circuit capacitance.

In one example, the geometry and film thickness for such thermally controlled components for wearable transponder tags is determined for temperature sensitivity for human monitoring. An exemplary structure may include an inductive antenna, L_{R}, in a circuit similar to that of FIG. 12A. In another exemplary structure, C_{R} could be replaced with a temperature sensitive bi-layer structure. In yet another exemplary structure, physical misalignment of the loops or plates mentioned above could also be used to sense skin-surface pressure or motion due to subcutaneous blood flow, joint motion, and so on.

In yet another example, a transponder (e.g., a wearable tag as described above for modulating frequency and/or time of a received signal) may include electrodes configured for positioning adjacent the skin of a subject. For instance, a 2-lead electrode to detect ECG bioelectric potential may be included with a tag sensor for conveying 2-lead ECG data. While Doppler detection of heart activity (and respiration) relates to mechanical motion, ECG tracks electrical heart activity and therefore provides complimentary data. Combined Doppler and ECG data may provide more robust heart rate determinations.

In some examples, and applications, a transponder may be realized in a low-cost, disposable, easily applied package. An illustrative form includes an adhesive “Band-Aid” or “patch” type package as illustrated; however, various other suitable tags or body-sensors will be apparent to those of ordinary skill in the art, and depending on the particular application, need not be affixed to the skin of a subject (for example, they may be affixed to clothing or worn around the neck or wrist, etc.). Exemplary fabrication technologies for the various implementations may include thin- and thick-film polymers, electroplated contacts and RF conductors, micro/nano-machined bio-potential electrodes, and nanotechnology, MEMS, or other transducer components that could be integrated on flexible carriers or substrates.

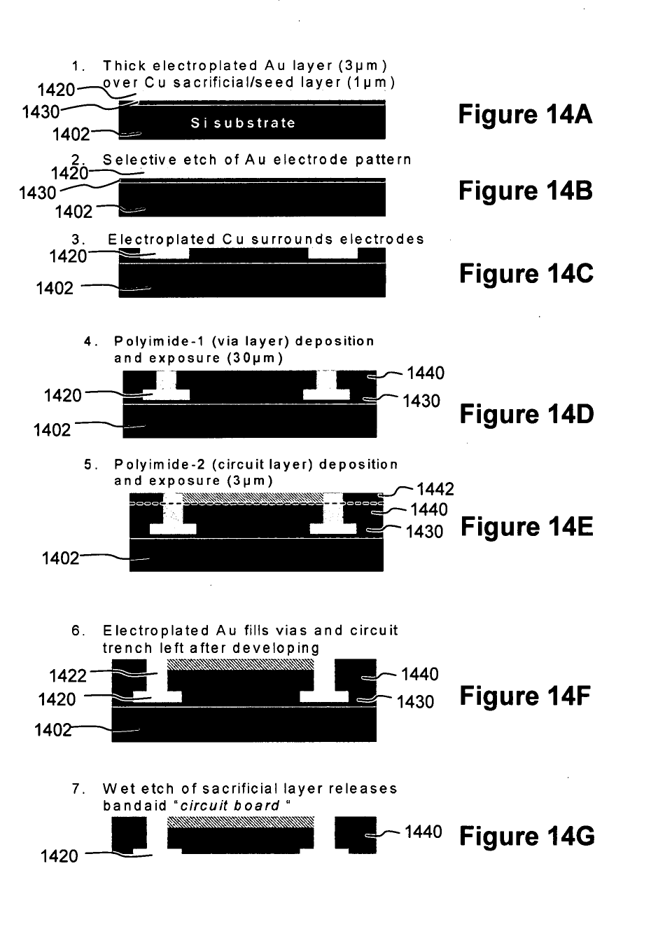

Exemplary transponder tags may be fabricated using well-known multi-layer and MEMS fabrication techniques. FIGS. 14A-14G illustrate an exemplary method for fabricating a transducer, in this particular method, an oscillating type transducer such as tag 1152 illustrated in FIG. 12A), and further including electrodes. In particular, the exemplary fabrication process includes fabricating electrodes and a circuit layer suitable for a “Band-Aid” tag.

Initially, an electroplated layer of conductive material 1420 (e.g., metal such as Au) is deposited over a sacrificial/seed layer 1430 (e.g., Cu) as illustrated in FIG. 14A. The conductive material is then patterned by any suitable method to form contact electrodes as illustrated in FIG. 14B. For example, a selective etch of the desired electrode pattern into conductive material 1420. The exposed seed layer 1430 is then plated (e.g., electroplated with a similar material such as Cu) to the height or thickness of the electrodes formed of conductive material 1420 as illustrated in FIG. 14C.

A layer of photosensitive polyimide 1440 is deposited over conductive material 1420 and seed layer 1430 via any suitable method. Photosensitive polyimide 1440 is further exposed to define vias between the electrodes and the circuitry as illustrated in FIG. 14D. A second, relatively thinner layer of polyimide 1442 is then applied, and exposed to define the metal pattern at the circuit level as illustrated in FIG. 14E. After developing the two-layer polyimide pattern, conductive material 1422 (e.g., Au) is electroplated to fill the mold defining the vias and metal circuitry in the polymide layers 1440 and 1442 as illustrated in FIG. 14F. Finally, the substrate 1402 and sacrificial seed layer 1430 (Cu) are removed, e.g., via etching, thereby releasing the polymer structure from the substrate as illustrated FIG. 14G.

It should be recognized that the exemplary method of fabricating a transducer is illustrative only and that many different methods may be used to fabricate the exemplary transducers as described. For example, various other semiconductor, MEMS, and nanotechnology processing techniques may be employed. Additionally, and in particular for transponders that include moving parts, e.g., MEMS components such as coils or fingers, may further be enclosed or housed in a more robust package (either for transport or during use).

Demodulators for Quadrature Receivers

According to another aspect of the present invention, various methods and systems are provided for demodulating received signals. In particular, exemplary linear and non-linear demodulation methods are described, as well as various exemplary rate-finding techniques such as fast Fourier transform (FFT), autocorrelation, and the like. The exemplary demodulation methods and systems are generally applicable to quadrature receivers and may be employed with any Doppler radar systems (including, e.g., those illustrated in FIGS. 2A, 2B, 2C, 4, and 5).

With reference again to FIG. 2A, a direct-conversion Doppler radar with an analog quadrature receiver is illustrated. Alternatively, quadrature mixing can be performed in digital domain, e.g., as illustrated FIG. 2C. As previously described, for an exemplary continuous wave (CW) radar and transmitting a single tone signal at frequency ω, a transmitted signal is reflected from a subject at a nominal distance d, with a time-varying displacement given by x(t). The baseband received signal at a single antenna system with quadrature receivers can be written as
r(t)=Aexp(j(ux(t)+θ))+k+w(t) (11)

where, u=ω/c=2π/λ, w(t) is the noise and θ is a phase offset. The signal x(t) is a superposition of the displacement of the chest due to respiration and heartbeat. The DC offset k is generally due to reflections from stationary objects and therefore generally does not carry information useful for sensing physiological motion; accordingly, the DC offset can be removed prior to quantization. A further reason for this is that the heartbeat is a very weak signal such that quantizing the whole signal generally requires a relatively high precision quantizer.

After sampling, the received signal can be written as
r[n]=Aexp(j(ux[n]+θ))+k+w[n]

We will assume that the noise w[n] is iid circular Gaussian.

Broadly speaking, an exemplary linear demodulator may then be of the form
\hat{x}[n]=arᵣ[n]+brᵢ[n]

where rᵣ[n] is the real part of the received signal, and rᵢ[n] the imaginary part.

An exemplary method for deriving a linear demodulator is now described. In this particular example, a linear demodulator is derived (and optimized) for an instance were the signal x[n] is considered to have symmetric distribution around its mean. In this example, two criteria are examined for optimization. First, maximization of the signal to noise ratio (SNR)

$${max}_{a , b} \frac{var [ aA cos ( υ x [ n ] + θ ) + bA sin ( υ x [ n ] + θ ) ]}{{a}^{2} var [ {w}_{r} [ n ] ] + {b}^{2} var [ {w}_{i} [ n ] ]}$$

Second, minimization of the mean square error (MSE)

$${min}_{a , b} E ⌊ ( \hat{x} [ n ] - x [ {n [ )}^{2} ⌋$$

To solve the optimization problems, assume first that the coordinate system is rotated with the angle −θ, i.e., a new set of coordinates are

$$[ \begin{matrix}{\tilde{r}}_{r} [ n ] \\ {\tilde{r}}_{i} [ n ]\end{matrix} ] = Q [ \begin{matrix}{r}_{r} [ n ] \\ {r}_{i} [ n ]\end{matrix} ]$$

where the orthogonal matrix Q denotes rotation with −θ. In this new coordinate system
\tilde{r}ᵣ[n]=A cos(ux[n[)+\tilde{w}ᵣ[n]
\tilde{r}ᵢ[n]=A cos(ux[n[)+\tilde{w}ᵢ[n]

where ((\tilde{w}ᵣ[n], \tilde{w}ᵢ[n]) is still white circular Gaussian noise. It follows that
var[aA cos(ux[n])+bA sin(ux[n])]=a²A²var[ cos(ux[n])]+b²A²var[ sin(ux[n])]

with the symmetry assumption on x[n] to show that cov(cos((ux[n]), sin(ux[n]))=0. Since var[sin(ux[n])>var[cos(ux[n]), the SNR maximizing solution can be given by a=0, b=1, i.e.,
\hat{x}[n]=\hat{r}ᵢ[n]

It can similarly (using the symmetry assumption) be shown that this solution also minimizes the MSE. What remains is to determine the matrix Q without knowing θ. Note that the covariance matrix of the rotated signal is given by

$$cov [ [ \begin{matrix}{\tilde{r}}_{r} [ n ] \\ {\tilde{r}}_{i} [ n ]\end{matrix} ] ] = [ \begin{matrix}{A}^{2} var [ cos ( υ x [ n ] ) ] + {σ}^{2} & 0 \\ 0 & {A}^{2} var [ sin ( υ x [ n ] ) ] + {σ}^{2}\end{matrix} ]$$

where again, cov(cos(ux[n]), sin(ux[n]))=0, is used. Thus, the matrix Q diagonalizes the covariance matrix. The unique matrix diagonalizing the covariance matrix is the matrix of eigenvectors, and therefore the optimum linear demodulator is projected unto the eigenvector corresponding to the largest eigenvalue. For example, if the signal ux(t) is small the signal model (2) is approximately linear in ux(t) and the method reduced to finding the signal subspace, which is optimum. Note this includes a linear approximation; the exact covariance matrix of the received signal is of course not known, but can be estimated by the empirical covariance matrix, and the eigenvectors of this used.

An exemplary method for deriving a non-linear demodulator is now described. Broadly speaking, a non-linear demodulator may be of the form
\hat{x}[n]=Arg(r[n]−k)/u

To implement the non-linear demodulator, however, k also needs to be known or estimated. This may be considered as a joint estimation problem of the parameters (A, k, x[n], n=0 . . . N−1); for this exemplary estimation it is assumed that x[n] is deterministic. It will be recognized by those of ordinary skill in the art that the estimation problem is invariant to rotations such that the measurements can be rotated so that k is real, e.g., by the matrix Q discussed above. Consider first a Maximum Likelihood (ML) estimation—where, given k, the ML estimator of the remaining parameters is

$$\hat{x} ( k ) [ n ] = Arg ( r [ n ] - k ) / υ$$

$$\hat{A} ( k ) = \frac{1}{N} {\sum }_{n = 0}^{N - 1} Re \{ ( r [ n ] - k ) exp ( - ⅈυ \hat{x} ( k ) [ n ] \}$$

The estimation problem for k can now be stated as

$$\hat{k} = {arg min }_{k ∈ R} d ( k )$$

$$d ( k ) = {\sum }_{n = 0}^{N - 1} {\vert r [ n ] - \hat{A} ( k ) exp ( j \hat{x} ( k ) [ n ] ) - k \vert}^{2}$$

Unfortunately, a closed form expression for k does not exist; furthermore, as will be outlined next, numerical solutions for k are generally difficult. A performance measure of an estimator includes the MSE of the estimate of x, m(k)=E[(\hat{x}(k)[n]−x[n])²]. To evaluate performance it can be assumed that um[n] is uniformly distributed over an arc [−θₘ, θₘ], however a numerical optimization algorithm for k is extremely hard to find as there are multiple local minima. On the other hand, as long as the estimate for k is sufficiently small, the MSE is quite insensitive.

An exemplary heuristic estimator may be used as a performance measure of the exemplary linear and non-linear demodulators for different systems. For example, suppose two points A and B on a circle. A line through the middle point of the cord between A and B then goes through the center of the circle. With the assumption that the circle center is on the X-axis, the intersection of this line with the X-axis gives k. So, assuming there is no noise, an estimate of k can be found as follows, by writing the above out in formulas

$$\hat{k} ( {n}_{1} , {n}_{2} ) = \frac{{\vert r [ {n}_{1} ] \vert}^{2} - {\vert r [ {n}_{2} ] \vert}^{2}}{2 Re \{ r [ {n}_{1} ] - r [ {n}_{2} ] \}}$$

for two arbitrary points r[n₁], r[n₂]. Since the signal is noisy, however, some kind of statistic has to be applied. As r[n₁]−r[n₂] can be near to zero, and extreme values of k therefore result, it can right away be predicted that the average of the estimated k values is a poor statistic, and simulations confirm this. Instead, the median of the estimated k values may be used, i.e., the exemplary estimator is then

$$\hat{k} = {median}_{{n}_{1} \neq {n}_{2}} \{ \hat{k} ( {n}_{1} , {n}_{2} ) \}$$

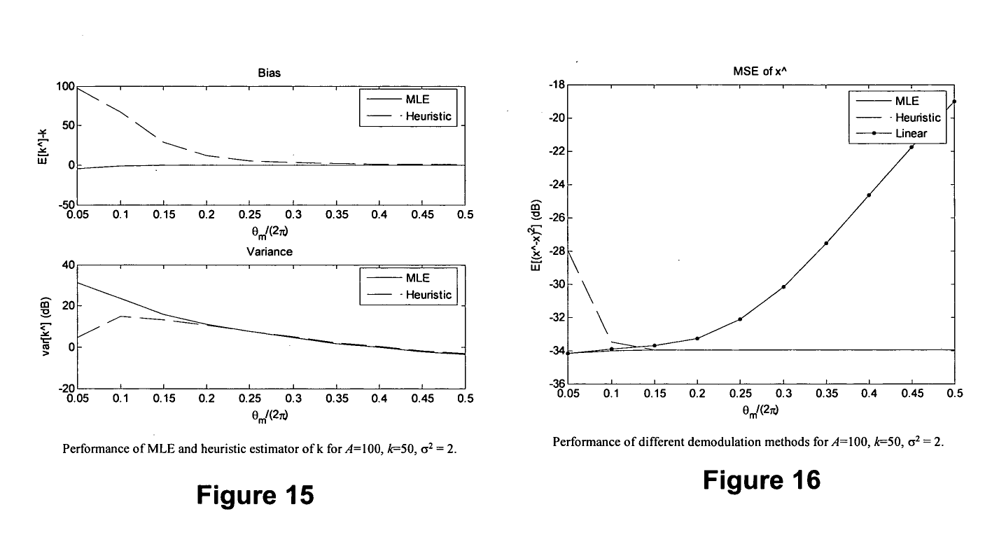

FIGS. 15 and 16 illustrate exemplary performance data of different demodulation methods compared on the basis of the ratio A²/σ² (which could be considered a “passband SNR”) and the maximum arc length θₘ. In particular, the performance is illustrated for a specific SNR. In all cases, N=100 samples are used for estimation. As performance measure for the estimation of x[n] the following modified MSE was used:

$$m = {min}_{a} E [ {( a \hat{x} - x )}^{2} ]$$

As illustrated for low arc length, linear demodulation performance is comparable to non-linear demodulation with ML estimation of k, but better than with the heuristic estimator (mainly because of the bias of this). Accordingly, varying SNR results in moving the point where one demodulator becomes better than the other.

A received signal after demodulation (linear or non-linear) may still be a relatively noisy signal compared to ECG signals, which typically have well-defined peaks. As such, conventional methods for ECG signal processing are generally not applicable to the received Doppler signals. Accordingly, various exemplary methods for finding heart rates and/or respiration rates from demodulated signals (whether via a linear or non-linear demodulator) include a fast Fourier transform (FFT), autocorrelation, and determining the time of the peaks, similar to the technique commonly used to find the heart rate from the ECG, but after heavy lowpass filtering.

In one example, the FFT can be calculated in a sliding window, and the peak of the FFT within a physiologically plausible range can be used to determine the rate of the heart signal. The autocorrelation function may be used to emphasize the periodic patterns in the windowed time domain signal. In one example, the autocorrelation function is calculated in a window, the local maxima are identified, and the time shift of the greatest local maximum between high and low physiologically plausible periods is taken to be the period of the windowed signal.

The selection of the window length is a tradeoff between the time resolution and the rate resolution. In one exemplary method, the window length is selected to include at least 2 periods of the signal, but not too long such the rate of the signal is likely to significantly change within the window length time.

Measurement Methods for I/O Imbalance Factors

In quadrature Doppler radar systems the I and Q receiver channels are typically subject to amplitude and phase imbalance factors caused by circuit and component imperfections. This may result in undesired linear transformation of the baseband output signals, which may degrade output accuracy and sensitivity. With known imbalance factors, compensation can be achieved through digital signal processing, but accuracy is generally affected by circuit modifications required to determine those factors. Accordingly, in one example provided herein, a method for measuring I/Q imbalance factors comprises introducing a phase shifter at the receiver input to simulate an object approaching with constant velocity, resulting in sinusoidal outputs which can be easily compared to determine phase and amplitude imbalance. The exemplary method can be performed without significant modification to the receiver system, allowing for more precise imbalance correction to be achieved.

An exemplary receiver includes a voltage controllable phase shifter inserted in either the LO or RF path. Linearly increasing the control voltage results in each channel output becoming a sinusoidal wave at the Doppler frequency with a phase delay corresponding to its path delay. Therefore, by comparing sinusoidal I and Q outputs, the imbalance factors between channels can be determined. Further, in some examples, the method does not require modification of the receiver (e.g., does not require radar circuit board modification), and the same source is used to supply RF and LO signals as is the case in Doppler radar direct-conversion systems. Further, in one example, measured imbalance factors can be compensated for with a Gram-Schmidt procedure to produce two orthonormal outputs (the Gram-Schmidt procedure is described in greater detail below and, for example, in “Compensation for phase and amplitude imbalance in quadrature Doppler signals,” Ultrasound Med. Biol., vol. 22, pp. 129-137, 1996, which is incorporated herein by reference).

With reference again to FIG. 2A an exemplary quadrature Doppler radar system is illustrated for sensing physiological motion in a subject. In one example, some or all components of the system shown in FIG. 2A may be included on a common board or device. Difference in circuit components between I and Q mixers and signal paths of the system, as well as inaccuracy of the 90 degree power splitter, may contribute to phase and amplitude imbalance. In particular, differences may create an undesired linear transform on the I and Q output signal components, thereby adversely affecting the orthonormal properties assumed for a quadrature receiver system. The baseband signal for each channel can be expressed as:

$${B}_{I} = A sin ( θ + p ( t ) ) \\ {B}_{Q} = {A}_{e} A sin ( \frac{π}{2} + θ + {ϕ}_{e} + p ( t ) ) , \qquad ( 12 )$$

where Aₑ and φₑ are the amplitude and phase imbalance factors, θ is constant phase delay for the traveling wave, and p(t) is the Doppler modulated signal.

It is possible to correct for a known phase and amplitude imbalance by a simple transformation known as the Gram-Schmidt procedure, shown in equation (13), which produces two orthonormal vectors.

$$[ \begin{matrix}{B}_{I\_ort} \\ {B}_{Q\_ort}\end{matrix} ] = [ \begin{matrix}1 & 0 \\ - tan {ϕ}_{e} & \frac{1}{{A}_{e} cos {ϕ}_{e}}\end{matrix} ] [ \begin{matrix}{B}_{I} \\ {B}_{Q}\end{matrix} ] \qquad ( 13 )$$

Imbalance factor measurements for a quadrature receiver system can be made by injecting two sinusoidal waves with slightly different frequencies to the LO and transmitter path respectively, using two external sources. However, in the case of the system similar to that shown in FIG. 2A, a major hardware modification to perform such a measurement, including a bypass of the LO, and removal of the antenna and the circulator may be needed.

In one example described here, an external voltage controllable phase shifter is connected between the antenna and the circulator/“antenna out” of the receiver system to provide similar conditions to those achievable through the use of two external sources, but without modification to the original system, thereby creating conditions similar to those in a practical homodyne radar system where the same source is used to produce both the RF and LO signals.

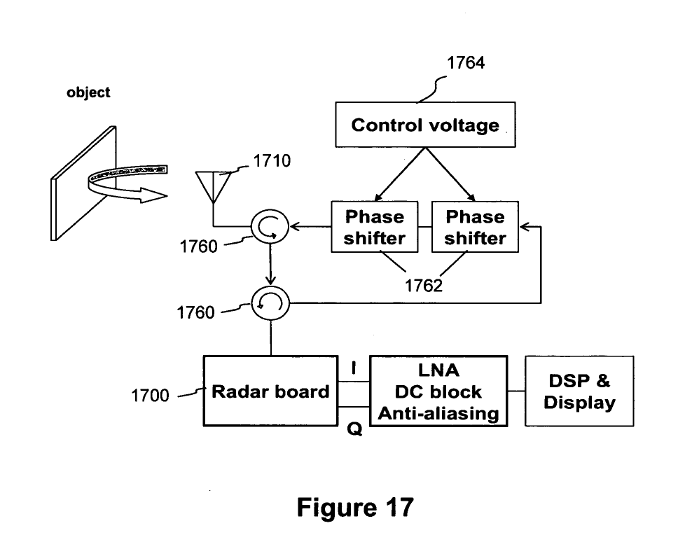

An exemplary imbalance measurement system is illustrated in FIG. 17. In this example, two external circulators 1760 and phase shifters 1762 are connected between a radar board system 1700 including a receiver (e.g., similar to that of FIG. 2A) and the antenna 1710. An object (e.g., a metal plate) is placed at a fixed distance in front of the antenna beam, while phase shifters 1762 simulate the phase delay that would result from an object moving at a constant velocity. According to Doppler radar theory, when a transmitting signal is reflected from an object with constant velocity, vᵣ, the frequency of the reflected signal, R_{receive}(t) is shifted by a Doppler frequency, f_{d}, where the polarity of the Doppler frequency is dependant on the direction of subject's velocity with respect to the radar.

$${R}_{receive} ( t ) = {A}_{r} cos [ 2 π {f}_{o} ( 1 \pm \frac{2 {v}_{r}}{c} ) t - \frac{4 π {d}_{o}}{λ} - {ϕ}_{channel} ]$$

where fₒ is carrier frequency,

$${f}_{d} = \frac{2 {v}_{r} {f}_{o}}{c} ,$$

dₒ is nominal distance of an object and φ_{channel} is phase delay caused by channel path length.

After mixing with the LO signal, a quadrature receiver produces sinusoidal outputs at the Doppler frequency, f_{d}, with a phase delay due to channel's path length.

$${B}_{I} ( t ) = {A}_{I} cos [ 2 π {f}_{d} t - \frac{4 π {d}_{o}}{λ} - {ϕ}_{I\_channel} ]$$

$${B}_{Q} ( t ) = {A}_{Q} cos [ \frac{π}{2} + 2 π {f}_{d} t - \frac{4 π {d}_{o}}{λ} - {ϕ}_{Q\_channel} ]$$

Amplitude and phase imbalance factors can then be measured by comparing these I and Q single frequency sinusoidal outputs.

In one example, the voltage controllable phase shifters 1762 are used with a fixed reflecting subject to simulate an object moving toward the radar with constant velocity, thereby creating an endless linear phase change in the reflected signal's path. This phase change may be realized by controlling the phase shifters 1762 with voltage from control voltage 1764 that is linearly incremented until the phase delay becomes 360 degrees, and then restores the voltage to a virtually identical 0 degree phase delay. In this manner a sawtooth wave with a peak-to-peak value corresponding to phase shifter's 360 degree phase delay can be used as a control voltage for generating the phase response of a continuously approaching object with constant velocity. The Doppler frequency, which is the frequency of the baseband output signals, can be determined by the slope of the sawtooth wave and equals to v_{2π}/t_{d}, where t_{d} is one period of the sawtooth wave, and is equal to the peak value of the wave that achieves 360 degrees of phase delay.

In one example, a Pulsar ST-21-444A commercial coaxial phase shifter is used for an imbalance measurement as described. The exemplary phase shifter is linear up to about 180 degrees, corresponding to 3.1 volts. In this example, two identical phase shifters were connected serially in the RF-out path (to ensure the system could fully produce the half-cycle of baseband output signal under linear phase control, e.g., to avoid approximations), and a sawtooth control voltage with a 3.1 volt peak-to-peak value was applied. The period of the sawtooth wave may be set to 1 second in order to get sinusoidal waves with a frequency of 1 Hz at each channel output, which approximates a heart rate signal.

In one example, the exemplary phase shifter and method is applied to a radar circuit board comprising a radar transceiver fabricated with surface mount components on a 10.2 cm by 11.2 cm FR4 substrate. The antenna may include a commercially available Antenna Specialists ASPPT2988 2.4 GHz ISM-band patch antenna. Further, Mini-Circuits JTOS-2700V VCO, RPS-2-30 two-way 0° power splitters, QCN-27 two-way 900 power splitter, and SKY-42 mixers may be used for the components of the radar board. The baseband output signals are further amplified by a factor of about 100 and filtered from 0.3 to 3 Hz with Stanford Research SR560 LNA's and digitized with a Tektronix 3014 digital oscilloscope.

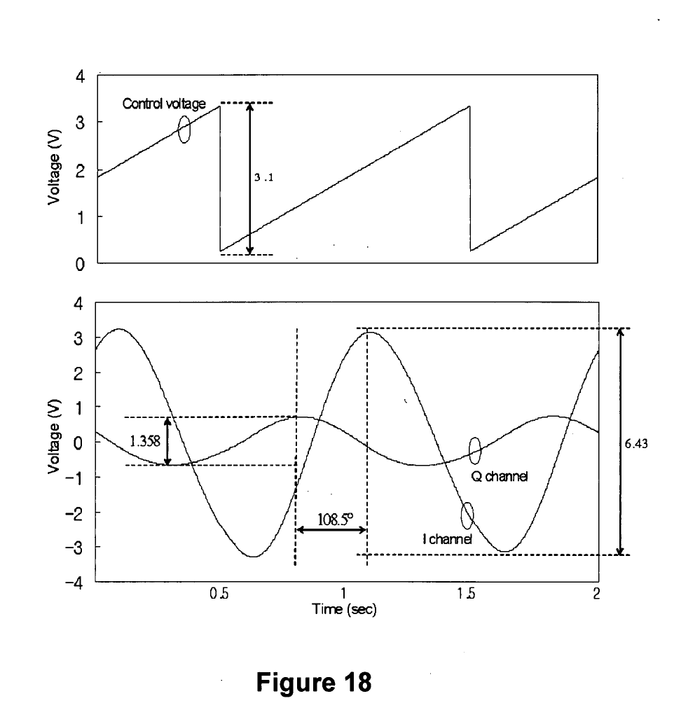

FIG. 18 illustrates data for an exemplary phase shifter control voltage with an amplitude of 3.1 volts and resulting I and Q sinusoidal outputs at a Doppler frequency of 1 Hz. The period of the I and Q sinusoidal waveforms corresponds the subject velocity simulated by the sawtooth control voltage. By comparing amplitude and phase delay of I and Q waveforms, the measured amplitude and phase imbalance factors may be determined; in this illustrative example, determined as 4.7 and 18.5 degrees, respectively.

Accordingly, the exemplary method and system described provides a measure of I/Q imbalance factors of a quadrature receiver. The I/Q imbalance factors may then be compensated for in future measurements; for example, via transformations such as the Gram-Schmidt procedure.

Arctangent Demodulation (w/DC Offset) for Quadrature Receivers

One challenge in providing robust Doppler radar sensing is detection sensitivity to a subject's position due to the periodic phase relationship between the received signal and local oscillator, resulting in “optimum” and “null” extreme subject positions. A quadrature Doppler radar receiver with channel selection has been proposed to alleviate such problems by selecting the better of the quadrature (I and Q) channel outputs, and is thus limited to the accuracy of a single channel. A frequency tuning technique with double-sideband transmission has also been proposed for Ka-band radar; however, such techniques generally involve more complex hardware with a tunable intermediate frequency.

According to one example, quadrature outputs (i.e., I and Q) are combined using full quadrature (arctangent demodulation) methods. Further, in one example, the quadrature outputs are combined with DC offset compensation. Arctangent demodulation may overcome position sensitivity issues while removing small-angle limitations on the range for phase deviation detection, which can be significant in single-channel systems operating at high frequencies. The additional use of DC offset compensation and exemplary center tracking methods may reduce or eliminate unwanted DC components produced by receiver imperfections and clutter reflections, while DC information required for accurate arctangent demodulation can be preserved.

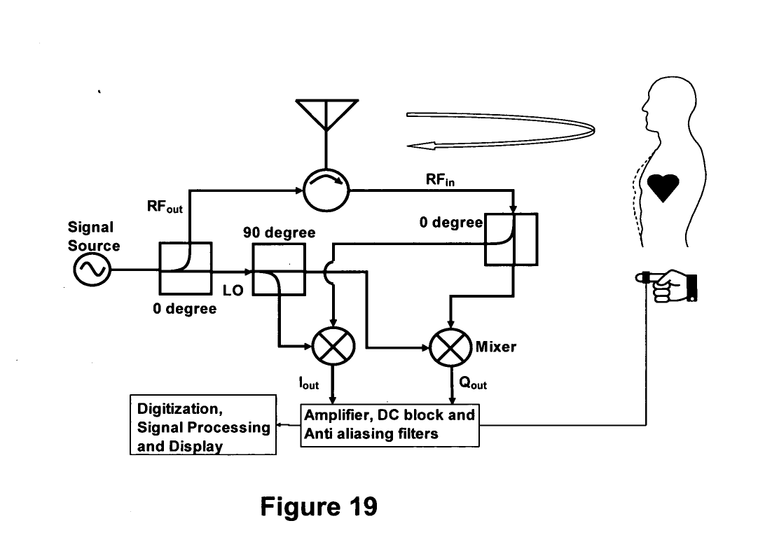

Initially, “null” and “optimum” conditions are described, as well as a discussion of quadrature channel imbalance and DC offsets. Typically, a Doppler radar system, e.g., as illustrated in FIG. 19, transmits a continuous wave signal, and phase-demodulates the signal reflected from a subject. A stationary human body reflects two independent time varying sources of motion with zero net velocity, resulting from respiration and cardiac activity, and the largest reflection of incident RF power occurs at the body surface. In terms of phase demodulation, the two extreme cases, “null” and “optimum”, occur periodically for subject positions at each λ/4 interval from the antenna, with λ/8 separation between null and optimum points. The mathematical basis for Doppler-radar sensitivity to a subject's position for a single-channel receiver are known, where in the optimum case the demodulated phase variation is linearly proportional to chest displacement, assuming the subject displacement is small compared to %. However, in the null case the demodulated heart and respiration related phase data can be self- or mutually-coupled, resulting in large detection errors.

Quadrature channel imbalance and DC offset issues are known in direct conversion receivers for radar and communications applications. With known channel imbalance factors, the Gram-Schmidt procedure can be used to correct imbalance errors as described above. Additionally, several DC offset compensation techniques have been described here, where the DC signal is assumed to be undesired (or at least contain little information). Accordingly, in one example, DC offsets are removed by using a high-pass filter, however, several modulation methods, including the exemplary arctangent demodulation method, contain “DC information” which is distinguished from unwanted “DC offsets” caused by imperfections in circuit components and reflections from stationary objects. For example, the DC information component, associated with subject position in Doppler radar, is typically several orders of magnitude larger than the amplitude of the periodic baseband signal related to heart activity, making it impractical to simply digitize the full signal with reasonable resolution.

Exemplary arctangent demodulation methods described here include techniques for isolating DC offset, DC information, and the ac motion signal to overcome dynamic range limitations for pre-amplifiers and analog to digital converters (ADC), without discarding important components of the desired data. Results of arctangent demodulation experiments with a subject at several different positions are also described, demonstrating proper preservation of cardiopulmonary-related motion information, and verifying accuracy insensitivity to subject position. In each example, the heart rate obtained from combined quadrature outputs agreed with a wired reference, with a standard deviation of less than 1 beat per minute. For the same measurements the standard deviation of data from each I or Q channel varied from 1.7 beats per minute in the optimum case, to 9.8 beats per minute in the null case, with the additional problem of heart rate tracking drop-outs in the latter case.

Initially, an exemplary quadrature receiver is described with respect to FIG. 19 (and which is similar to quadrature receivers described previously), which illustrates the block diagram of a quadrature Doppler radar system, wherein a single signal source provides both the RF output and LO signals. The LO signal is further divided using a 90° power splitter to provide two orthonormal baseband outputs. Assuming that heart and respiration motion is given by x(t) and y(t), the quadrature baseband outputs can be expressed as

$$\begin{matrix}{B}_{I} ( t ) = sin [ θ + \frac{4 π x ( t )}{λ} + \frac{4 π y ( t )}{λ} + Δϕ ( t ) ] , & ( 14 ) \\ and & \\ {B}_{Q} ( t ) = cos [ θ + \frac{4 π x ( t )}{λ} + \frac{4 π y ( t )}{λ} + Δϕ ( t ) ] & ( 15 )\end{matrix}$$

where Δφ(t) is the residual phase noise, and θ is the constant phase shift related to the nominal distance to the subject including the phase change at the surface of a subject and the phase delay between the mixer and antenna.

The null and optimum cases for the output signal with respect to θ can be observed in (14) and (15). When θ is an odd multiple of π/2, the baseband signal of the Q channel is at an optimum point while that of the I channel is at a null point. On the other hand, when 9 is an integer multiple of π, the baseband signal of the I channel is at an optimum point while that of the Q channel is at a null point. Assuming that both x(t) and y(t) are much smaller than λ/4π (the small angle approximation) and that they can be simplified as sinusoidal waves of frequency f₁ and f₂, with θ an integer multiple of π, (1) and (2) become
B₁(t)≈A sin 2πf₁t+B sin 2πf₂t+Δφ(t) (16)
B_{Q}(t)≈1−[A sin 2πf₁t+B sin 2πf₂t+Δφ(t)]² (17)

Where f₁<<f₂ and A>>B. Note that the small angle condition becomes more challenging as λ decreases. In this case the “optimal” I channel output is linearly proportional to chest motion and it should be possible to obtain the desired data accurately, with appropriate filtering. The “null” Q channel output given by (17) can be expanded and rearranged as:

$${B}_{Q} ( t ) \approx 1 - \frac{1}{2} [ ( {A}^{2} + {B}^{2} ) - {A}^{2} cos 4 π {f}_{1} t - {B}^{2} cos 4 π {f}_{2} t - 2 AB ( cos 2 π ( {f}_{2} + {f}_{1} ) t - cos 2 π ( {f}_{2} - {f}_{1} ) t ) + 2 Δϕ ( t ) ( 2 A sin 2 π {f}_{1} t + 2 B sin 2 π {f}_{2} t + Δϕ ( t ) ) ] . \qquad ( 18 )$$

Several problematic phenomena can be observed for this “null” case from (18). There is a significant DC component present at the output, and the output is no longer linearly proportional to displacement. The square terms result in signal distortion either by doubling the signal frequency or by mixing heart and respiration frequencies, while the linear terms are multiplied by the residual phase noise, thus degrading the SNR.

An exemplary direct conversion quadrature-receiver Doppler radar system, similar to that shown in FIG. 19 and looking at each output channel independently may include the following components. A commercially available Antenna Specialists ASPPT2988 2.4 GHz patch antenna, with a gain of 7.5 dBi, an E-plane range of 65°, and an H-plane range of 80°. A Mini-Circuits JTOS-2700V VCO for a signal source, which delivers 0.8 dBm at 2.4 GHz to the antenna port. A Mini-Circuits RPS-2-30 for each two-way 0° power splitter, and a Mini Circuits QCN-27 for the two-way 90° power splitter. A Mini-Circuits SKY-42 for each mixer. As the measurement setup shown in FIG. 19 indicates, the baseband output signals are amplified (˜1000×) and band-pass filtered (0.03 Hz-10 Hz) with SR560 LNA's, and then digitized with a DT9801 ADC card. Heart and respiration rates may be extracted in real time with software based on an autocorrelation algorithm, for example, as described herein or in B. Lohman, O. Boric-Lubecke, V. M. Lubecke, P. W. Ong, and M. M. Sondhi, “A digital signal processor for Doppler radar sensing of vital signs,” IEEE Engineering in Medicine and Biology Conf., Istanbul, Turkey, October 2001, which is incorporated by reference.

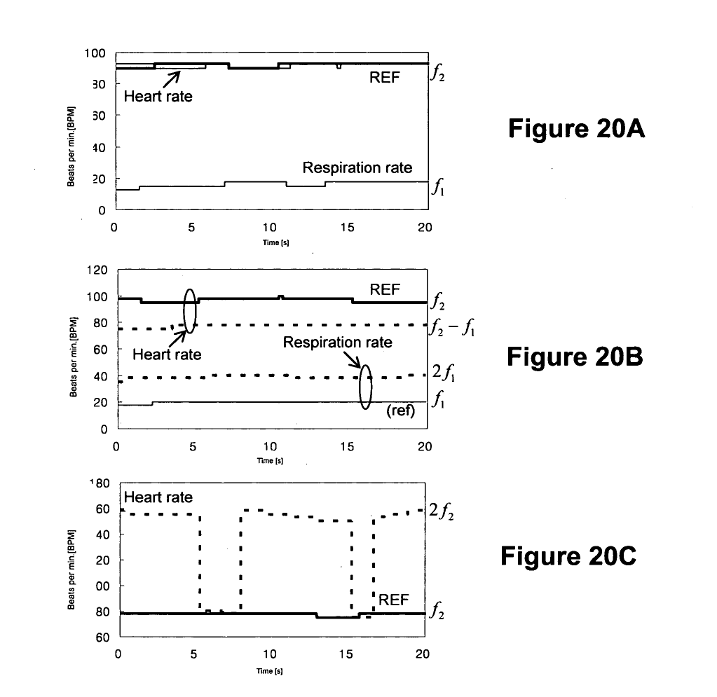

The heart rate may be compared with a reference obtained from a wired finger pressure pulse sensor (UFI 1010). Measurement results as described are illustrated in FIGS. 20A-20C, and the distortion discussed above observed. FIG. 20A corresponding to an “optimum” case, the baseband data is linearly proportional to the actual signal resulting in an output that corresponds well with the reference signal. FIGS. 20B and 20C illustrate the “null” case data taken both during continuous breathing, and breath-holding, respectively. As predicted in equation (18), FIG. 20B illustrates the detected heart rate decreased by an amount equal to the respiration rate, and a doubled respiration rate is evident in FIG. 20C.

Single receiver-channel Doppler radar system limitations described previously can be eliminated by using a quadrature receiver system like the one shown in FIG. 19, with both channels (e.g., I and Q) considered simultaneously. In particular, a quadrature receiver provides two orthonormal outputs, thus ensuring that when one channel is in a “null” position the other will be in an “optimum” position. Furthermore, by combining the two channels, accurate phase demodulation can be achieved regardless of the subject position or displacement amplitude, the latter being restricted to the small angle deviation condition for even the optimum case in a single channel receiver. As shown in equations (14) and (15), the I and Q outputs are the cosine and sine of a constant phase delay caused by the nominal distance to a subject, with a time varying phase shift that is linearly proportional to the chest displacement.

Applying an arctangent operation to the I and Q output data ratio, phase demodulation can be obtained regardless of the subject's position as

$$ϕ ( t ) = arctan ( \frac{{B}_{Q} ( t )}{{B}_{I} ( t )} ) = arctan ( \frac{sin ( θ + p ( t ) )}{cos ( θ + p ( t ) )} ) = θ + p ( t ) , \qquad ( 19 )$$

where

$$p ( t ) = \frac{4 π ( x ( t ) + y ( t ) )}{λ}$$

is the superposition of the phase information due to respiration or heart signals. However, quadrature channel imbalance and DC offset act as a linear transform on the I and Q components, thus modifying equation (19) to:

$${ϕ}^{′} ( t ) = arctan ( \frac{{B}_{Q} ( t )}{{B}_{I} ( t )} ) = arctan ( \frac{{V}_{Q} + {A}_{e} sin ( θ + {ϕ}_{e} + p ( t ) )}{{V}_{I} + cos ( θ + p ( t ) )} ) , \qquad ( 20 )$$

where vₜ and v_{Q} refer to the DC offsets of each channel, and Aₑ and φₑ are the amplitude error and phase error, respectively.

Correction for a known phase and amplitude imbalance is straight forward using the Gram-Schmidt procedure (for example, as described herein and in “Compensation for phase and amplitude imbalance in quadrature Doppler signals,” Ultrasound Med. Biol., vol. 22, pp. 129-137, 1996, which is incorporated herein by reference). The DC offset issue is generally more complex, however, due to the fact that the total DC signal may contain DC information for accurate demodulation. The DC offset is generally caused by two main sources: reflections from stationary objects (clutter), and hardware imperfections. Hardware imperfections include circulator isolation, antenna mismatch, and mixer LO to RF port isolation, resulting in self-mixing which produces a DC output. On the other hand, as indicated by equation (18), DC information associated with the subject's position is also part of each baseband signal. The magnitude of this DC level is dependent on the subject's position, such that the DC level is higher for subject positions closer to the “null” case. According, in one example of arctangent demodulation, the DC information is extracted from the total DC output and preserved (e.g., stored in memory).

An exemplary coaxial quadrature radar system, e.g., as shown in FIG. 19, may be used to examine arctangent demodulation issues. The same antenna, baseband pre-amplification, and data acquisition and heart rate extraction systems as previously described are used. Further, an HP E4433B signal generator serves as the LO and is divided into RF and LO signals by a Mini-Circuits ZFSC-2-2500 signal splitter. A Narda 4923 circulator isolates the transmit and receive signals, with the circulator RF to LO isolation measured to be −22 dB. The LO signal is further divided by a hybrid splitter, Narda 4033C, to provide quadrature outputs. A Mini-Circuits ZFM-4212 serves as the mixer in each channel. Amplitude and phase imbalance factors for the exemplary coaxial radar system as described were measured as 1.013 and 1°, respectively.

The DC offset caused by hardware imperfections may be measured by terminating the antenna port with a 50Ω load. The main contribution to the DC offset is caused by self-mixing with circulator leakage power, dependent on the phase difference between the LO and antenna feed line. By connecting a phase shifter between the LO feed line and varying the phase delay, the DC offset range for each channel may be measured at the corresponding mixer's IF port and, in one example, determined to be 19.4 mV for the I channel and 19.8 mV for the Q channel with an LO power of 0 dBm. The DC offset due to reflections may be estimated by putting an object, e.g., a large metal reflector, at a distance of 1 and 2 meters from the receiver, with a half-wavelength position variation to find the maximum and minimum DC values. The DC offset range for the I and Q channels from a reflector at 1 or 2 meters distance in this instance are 3 mV and 3.4 mV, and 0.6 mV and 0.8 mV, respectively. Accordingly, in this example, the DC offset is dominated by the contribution from imperfections in the circuit components rather than from clutter located 2 meters away from radar.

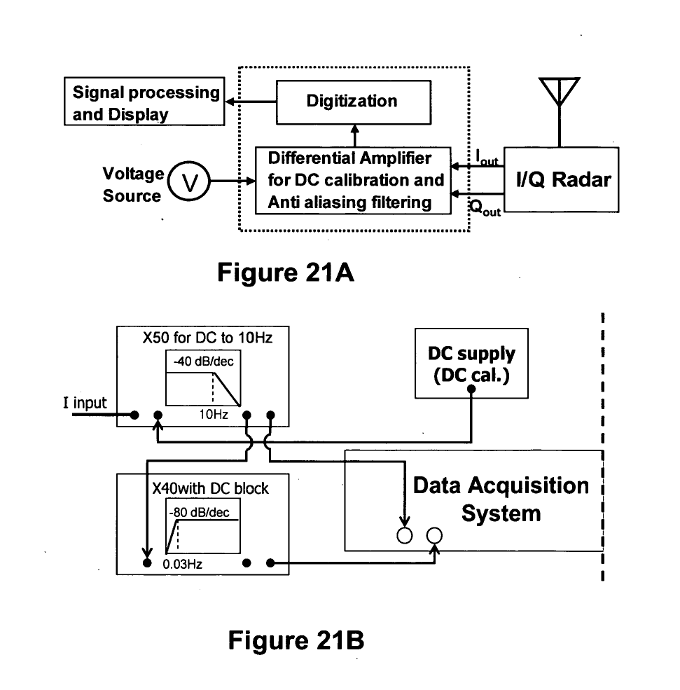

An exemplary measurement set-up for DC compensation is shown in FIG. 21A. In this example, a coaxial radar system as described previously and illustrate in FIG. 19 is used to collect data from a seated subject facing the antenna at a distance of about 1 meter. A wired finger pressure pulse sensor provides a reference for the heart rate. The DC offset components, which may be determined as described above, may be subtracted from the output signal.

Additionally, in one exemplary method and system to preserve the relatively large DC information level while sufficiently amplifying the weak time-varying heart-related signal is illustrated in FIG. 21B. With no object within 1 meter in front of the radar system, the internally or externally induced DC offset of each channel is measured. These DC offsets are then calibrated by using differential amplifiers, each with one input port connected to a DC power supply. The DC supplies are used to generate the same voltage as the DC offset of each channel, thus producing a zero DC level at the output. While preserving this condition, a subject is then located at a distance of about 1 meter from the radar, whereby the full DC level, including the heart motion signal, is detected at each channel. In one example, to achieve sufficient amplification of the signals, three amplifiers are used at the baseband stage of the I and Q channels. The first amplifier comprises a differential amplifier with a gain of 50 to amplify both the DC and the heart motion signal, and calibrated the DC offset. Subsequently, the output of the first amplifier is divided into two outputs, one of which is saved in the data acquisition system and the other saved after the DC is removed and the ac content is amplified. Two amplifiers are used for the DC blocking filter with a cut-off frequency of 0.03 Hz and gain settings of 20 and 2, respectively, in order to obtain a high-Q (−80 dB/dec) and thus a sharp cut-off.

Arctangent demodulation is then performed using the signals with and without DC content using Matlab software, for example. The signal with DC content was multiplied by 40 in the Matlab code before summation with the ac signal that was pre-amplified before the ADC. At the same time, the ac-only signal is filtered with a filter, e.g., a Butterworth filter, that passes frequencies between 0.9 to 2 Hz to reduce any still-detectable low frequency component due to respiration and avoid including this effect twice when summing with the DC-included signal. Consequently, a high-resolution heart motion signal combined with a virtual DC component is created. In an absence of the exemplary method, the DC component would likely saturate the amplifiers before the smaller heart motion signal could be sufficiently amplified for recording.

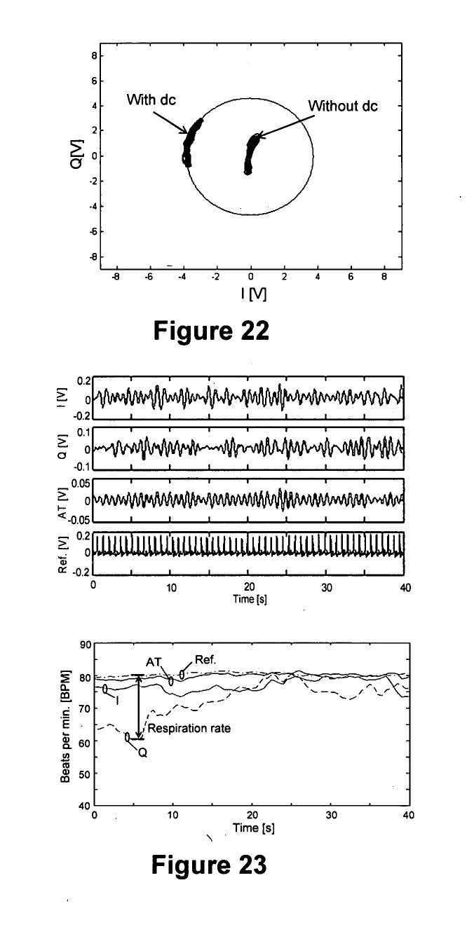

To verify that the DC information is properly preserved, I/Q data after imbalance and DC offset compensation may be plotted on a polar plot. For example, two orthonormal sinusoidal functions of the same phase information will compose part of circular trace centered at the origin, corresponding to the phase information. Exemplary data is illustrated in FIG. 22, where exemplary I/Q baseband signals DC information are plotted and form a part of an almost perfect circle centered at the origin, indicating that the DC information is correctly accounted for (it would be a circle for two orthonormal sinusoids). Additionally, the same measurement with the DC portion removed is also shown, appearing at the origin where the phase information cannot be recovered with the same certainty.

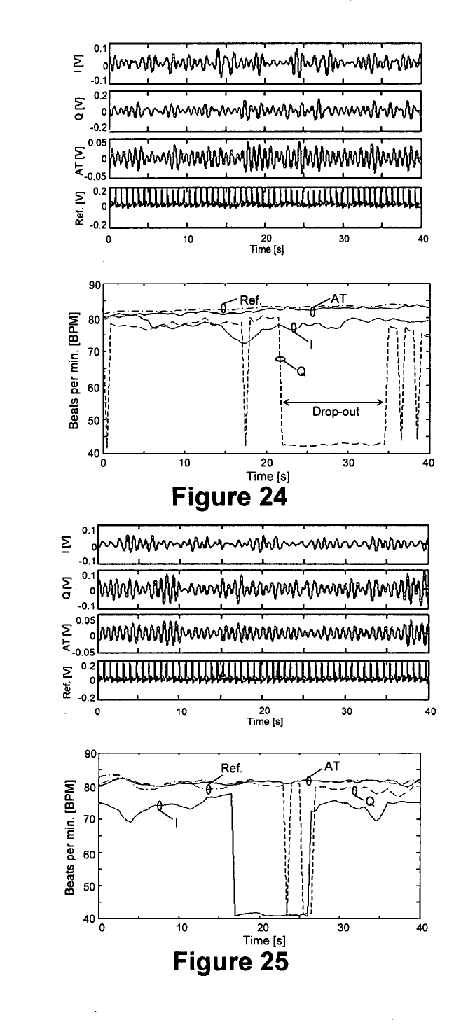

FIGS. 23 through 25 illustrate I, Q, and arctangent demodulated signals obtained using the exemplary measurement setup shown in FIGS. 21A and 21B for a subject in an intermediate position for both channels (FIG. 23), close to a null position for the Q channel (FIG. 24), and close to a null position for the I channel (FIG. 25). It should be noted, however, that the null and optimum positions cannot be set exactly for heart rate measurements, as the nominal distance (and associated phase) varies as a result of respiration and effects rate data accordingly. To illustrate the exemplary arctangent demodulation method, standard deviation was used to provide a quantitative comparison. As shown in FIGS. 24 and 25, a drop-out region occurs at the null point due to degradation in signal power, and this region is excluded when calculating standard deviation. In FIG. 23, the Q channel heart signal is affected by the presence of the respiration signal, which is around 20 BPM, at the beginning of the measurement interval. The I and Q channels show an error of 3.9 or 9.8 beats, respectively, during the 40 second time interval while the arctangent combined output has an error of only 0.95 beats. In FIG. 24, 35% of the Q channel data could not be acquired or, dropped out, and the rest has an error of 4.8 beats. The more stable I channel data still has an error of 5.2 beats, while the arctangent combined output has an error of only 0.9 beats. In FIG. 25, both I and Q channels drop out for 23% and 5% of the total time interval, respectively. The I channel data has an error of 7.5 beats and the Q channel data has an error of 1.7 beats, while the arctangent combined output has an error of only 0.6 beats. From the measurement results described above it is evident that arctangent demodulation results are significantly more accurate than any single channel output, with an error that is consistently less than 1 beat in standard deviation over the 40-second monitoring interval, and when using this data there is no drop-out region. Thus, arctangent demodulation produces robust and accurate data for rate tracking regardless of a subject's position, and typically without need for channel selection.

In another example, center tracking quadrature demodulation is described, including full quadrature (arctangent) detection and DC offset compensation. As described with respect to FIG. 22, for example, I/Q baseband signals can be plotted to form a part of an almost perfect circle. If there is large displacement of the subject and/or a relatively high frequency system, the center of the circle may be determined. Accordingly, in one example, an arc is extracted from the signal, movement is estimated to obtain an arctangent demodulation of the signal, and the center of the circle may be determined.

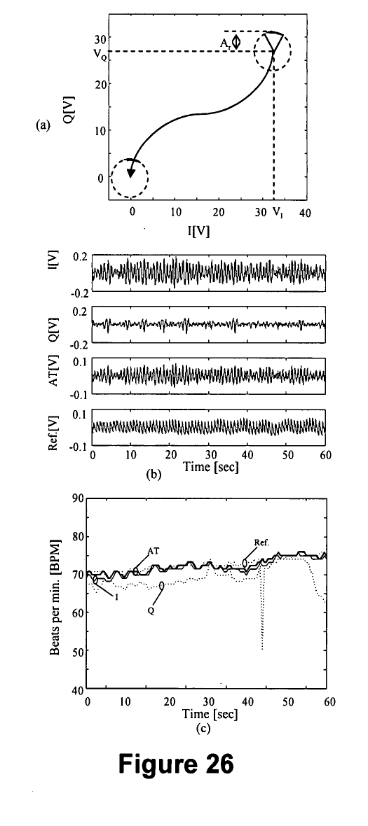

For example, as illustrated in FIG. 26 (the top portion thereof), when there is only one subject, and the reflected signal is phase-modulated by variation from the subject, complex plot of quadrature outputs therefrom forms fraction of the circle that has a radius of signal amplitude, Aᵣ, with center offset by DC offset of each channel. This property allows elimination of DC offset and preservation of DC information, which is the magnitude of the radius projected on each axis, if the center of arc formed by motion of a subject is tracked back to the origin of the complex plot. Arctangent demodulation of quadrature outputs, whose complex plot is centered at the origin, produces phase information which corresponds to actual motion of a subject, thereby allowing real time subject motion monitoring.

These properties can extend their validation to the larger phase modulated signal that happens when a subject's motion variation becomes bigger than wavelength of the carrier frequency. Complex plot of the I and Q outputs is related mainly with both received signal power and phase deviation due to a subject's motion. From (14) and (15), received signal power becomes Aᵣ², square root of which is the radius of the arc formed by phase deviation from a subject's motion. Phase variation, which is proportional to the arc length, is proportional to the ratio of subject's motion over wavelength of the carrier signal. In other words, arc length becomes longer either due to the increase of subject's actual motion or due to the increase of the carrier frequency. Consequently, when a subject is moving with large deviation resulting in changing received signal power, the radius of the arc will vary while the center is located at the same point, thus forming a spiral like shape rather then a circle. On the other hand, when operating frequency is increasing so that small physical motion of a subject is converted in large phase variation, longer arc length on a circle can be obtained.

An exemplary coaxial quadrature radar system and measurement set-up for DC compensation is illustrated in FIGS. 21A and 21B, respectively. In one example, data is collected from a seated subject facing the antenna at a distance of about 1 meter for the stationary subject data, and for tracking moving subject data is collected from a subject walking back and forth with 200 cm deviation from 100 cm away from the antenna. A commercially available Antenna Specialists ASPPT2988 2.4 GHz patch antenna is used, with a gain of 7.5 dBi, an E-plane range of 65°, and an H-plane range of 80°. An HP E4433B signal generator is used as the LO and divided into RF and LO signals by a Mini-Circuits ZFSC-2-2500 signal splitter. A Narda 4923 circulator is used to isolate transmit and receive signals, with the circulator RF to LO isolation measured to be −22 dB. The LO signal is further divided by a hybrid splitter, Narda 4033C, to provide quadrature outputs. A Mini-Circuits ZFM-4212 is used for the mixer in each channel. As described, to preserve the relatively large DC information level while sufficiently amplifying the weak time-varying heart-related signal without saturating neither pre-amplifiers nor ADC, two serially connected pre-amplifiers, SR560 LNAs, are employed. First amplifier has gain of 50 times from DC to 10 Hz in order to preserve DC information while second amplifier further amplifies by 40 times from 0.03 Hz to 10 Hz to provide more SNR to small cardiac signal. Each output is digitized with a DT9801 ADC card and saved in data acquisition system. Subsequently, those two outputs are combined together in Matlab after multiplication of DC included signal by 40 times to compensate amplification difference between both outputs. At the same time, the ac-only signal was filtered with a FIR, which has linear phase delay, Flat-Top filter that passed frequencies between 0.8 to 10 Hz to eliminate the still-detectable low frequency component due to respiration and thus avoid including this effect twice when summing with the DC-included signal. Consequently, high heart-related signal power with DC information can be obtained. The reconstructed DC included signals still require more signal processing to exclude DC offset caused by either clutter or leakage LO power in the system.

As previously described, chest motion from a subject forms an arc in the complex plot that is centered away from the origin by the amount of DC offset. Center estimation may be done before arctangent demodulation. For example, the first three seconds of data may be used for estimating center of arc, which can be one cycle of respiration and can form enough arc length. The center of the arc may be determined for each pair of points, and the results combined to get an improved estimate of the center, in one example, the median. Quadrature signals that form arcs centered at the origin in a complex plot are combined by using arctangent demodulation. Demodulated output may then be digitally filtered by a Flat-Top filter with frequency range of 0.8 to 10 Hz to obtain heart signal, with larger bandwidth sharper heart signal can be obtained. Heart rates may be extracted in real time with custom software based on an autocorrelation algorithm or the like, and heart rate may be compared with that obtained from a wired finger pressure pulse sensor (UFI 1010) used as a reference. Additionally, subject's movement tracking measurement also has been done with same arctangent demodulation method explained above. However, in this case since phase variation caused by a subject's motion is much bigger than 2π or half wavelength, which is 6.25 cm at 2.4 GHz, arctangent demodulated output need to be unwrapped and complex plot is no more small fraction of the circle but spiral like shape which has the same center point. This is to be expected, because DC offset caused by clutter or leaking within the device is fixed while receiving signal power which corresponds to the radius of the complex signal circle varies associated with a subject's distance from the antenna.

FIG. 26 illustrates the I, Q, and arctangent demodulated signals with an exemplary center tracking method. In this instance, a subject is at I channel in null position thus heart rate is modulated by respiration signal, since heart signal keep changing its polarity associate with nominal phase delay caused by chest position due to respiration, while Q channel is in optimum position resulting in higher accuracy then the other one. Arctangent result maintains accurate heart rate. Standard deviation is used to provide a quantitative comparison of accuracy. The I and Q channels show an error of 1.7 or 5.1 beats, respectively, during the 60 second time interval while arctangent combined output has an error of only 1.3 beats. Data obtained at several difference subject positions is also processed, Arctangent demodulation outputs always give better than or at least same accuracy as the better of I and Q channel outputs.

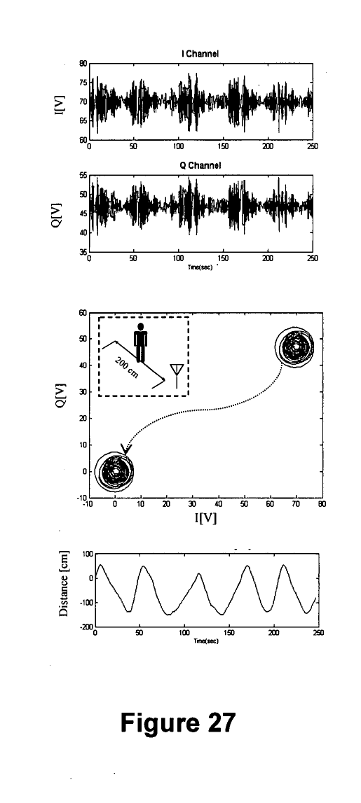

FIG. 27 illustrates a subject's movement tracking result by using Arctangent demodulation. For this measurement, a subject moves back and forth within 200 cm distance along the aligned line of the radar. As expected, complex plot forms different radius circles, due to the received signal power variation, with sharing same center point associate with DC offset. Arctangent output is phase information, which is linearly proportional to the actual distance variation, and converting coefficient should be multiplied to get distance information. From the lower plot, it is clear that coefficient of λ/4π multiplication can covert phase information to distance information.

Accordingly, exemplary arctangent methods are described, including DC compensation and center estimation methods. Exemplary methods enable restoring DC information signals directly from I and Q signals associated with subject's motion, which can compensate DC clutter caused by background stationary objects as well as additional DC information from other body parts of a subject. Moreover, detection accuracy limited within small phase variation range (e.g., as is the case in a single channel system) is no longer an obstacle as arctangent demodulation provides baseband output linear to subject motion regardless of phase variation range due to subject's motion. The exemplary method may track a moving subject's position though respiration or heart signal.

Data Acquisition System for Doppler Radar Systems

According to another aspect of the present invention, an exemplary data acquisition system (DAQ) is described. In one example, a system comprises analog to digital converters and automatic gain control (AGC) units for increasing the dynamic range of the system to compensate for the limited dynamic range of the analog to digital converters. In a two-channel quadrature receiver, for example, the quadrature signal may be analyzed using a suitable arctangent demodulation method as described herein for extracting phase information associated with cardiopulmonary motion, where arctangent demodulation of the two channels provides accurate phase information regardless of the subject's position.

Additionally, it is generally desired to extract and save DC information. For example, DC information, in addition to DC offset, is desirably recorded. A common concern in bio-signals such as EEG and ECG is baseline drift or wander. Slowly changing conditions in the test environment and in the subject can cause a drift outside of the contributions due to noise. For an exemplary direct conversion Doppler radar system, a baseline drift is a significant change in the DC component of the signal. This may depend on the distance of the subject and the orientation position of the subject which may change the radar cross section of the subject. Therefore, in one example, a system is operable to record a large DC offset that includes certain DC information, as well as a small time varying signal on top of the DC offset.

In one exemplary system for Doppler radar sensing of physiological motion of at least one subject includes an analog to digital converter, and an automatic gain control unit, wherein the analog to digital converter and the automatic gain control unit are configured to increase the dynamic range of the system, modifying the DC offset value and/or gain for the signal of interest. Modifying the DC offset value may include removing the DC offset alone; removing the DC offset, and adjusting and recording the gain; tracking and removing a DC offset value; modifying the DC offset value comprises removing and recording the DC offset, and adjusting and recording the gain; and the like (note that tracking extends to independent or concurrent DC and gain modifications). Additionally, the exemplary system may further adjust and recording the gain.

Various exemplary data acquisition methods and systems include recording a large DC offset as well as a relatively small time varying signal. Exemplary data acquisition methods and systems include a multi-band approach and a two-stage voltage reference approach. An exemplary multi-band system includes a low-pass and band-pass filters designed to have particular cross over points. In the case of the bio-signals for respiration and heartbeat, a likely crossover point between low-pass and high pass would be 0.03 Hz. For example, an anti-aliasing filter at 100 Hz provides two bands: DC −0.03 Hz band that records the DC offset and a 0.03 Hz to 100 Hz hand, which records cardiopulmonary activity. The low band is fed directly into a 16-bit ADC. The high band is sent through a VGA controlled by an AGC. This amplified high band is acquired by a second ADC. As long as the gain amount is properly recorded, an accurate reconstruction of the input signal can be made. The quantization noise introduced by the low band ADC may limit any improved dynamic range afforded by the VGA for the high band. Therefore, in one example, quantization errors introduced by the DC offset ADC is compensated for. The two stage voltage reference approach is similar to the multi-band, but also includes a DAC that supplies the recorded DC level to be used as a reference for the VGA. An advantage to this technique is that as the gain is increased for the second stage the dynamic range of the system also increases. This occurs because quantization errors introduced by the first ADC is compensated for as gain is increased in the VGA.

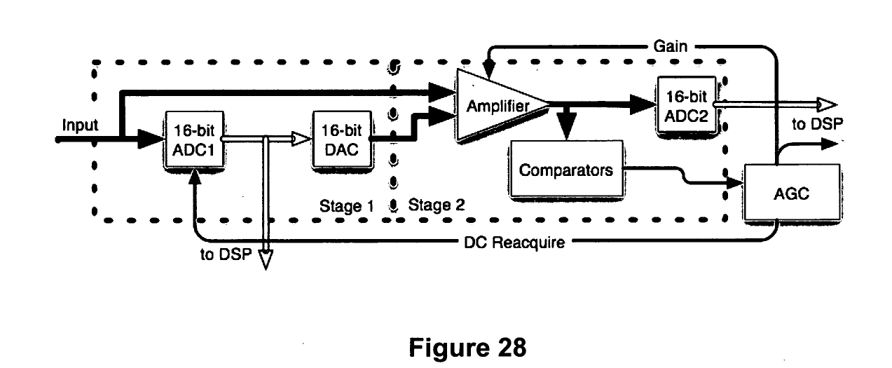

In another example, a DAQ system is comprised of two signal stages and an AGC unit as seen in FIG. 28. The first signal stage includes a 16-bit ADC (ADC1) and a 16-bit DAC, which acquires an estimated value of the DC offset and provides the reference level for the second stage. The second signal stage includes a VGA and another 16-bit ADC (ADC2), which includes a set of comparators to provide gain control feedback for the AGC. The second stage is responsible for acquiring the cardiopulmonary motion.

Input to the first signal stage includes the large DC offset as well as the small signal that provides the important cardiopulmonary motion information. A fixed gain pre-amplifier is used to provide proper signal amplitude out of the RF mixer. At the start of the acquisition cycle, ADC1 instantly acquires a value from the signal. This value is the initial estimated DC offset. This initial value is given to the DAC and the DAC is instructed by the AGC to output the same value.

The second stage uses the estimated DC offset from the DAC as a reference voltage level in difference with the input signal from the pre-amp. The reason for using the DAC to recreate the DC offset is to compensate for quantization errors in ADC1. In the beginning of the cycle, the gain of the VGA is at the lowest setting. Comparators at the output check for signal over-shoot. A second set of comparators also checks a voltage window for gain increase. If a signal remains within the gain window for a set amount of time (2 respiratory cycles or about 4 seconds), the gain of the VGA is increased by a step. A condition of signal over-shoot will cause the AGC to request ADC1 to reacquire a new DC value and send it on to the DAC. In addition, the VGA is returned to its lowest gain value and the acquisition cycle is restarted.

Generally, AGC units perform best with continuously variable gain amplifiers. These VGAs adjust depending on the signal strength to provide the highest possible dynamic range. However, due to the need to record the DC offset, it is important to maintain the relationship between the DC and the small signal. Therefore, a digitally controlled amplifier is needed. In one example, a dB linear gain scale is utilized with the highest number of steps possible.

There are two methods to optimize the reference voltage level. One uses a low pass filter to find an average value and the other utilizes median value finding algorithms that may be accomplished through Matlab or an FPGA (field programmable gate array). Optimization of the reference level is valuable for this particular method of data acquisition because the initial estimated DC offset may not be the best possible for improving dynamic range. For example, a simple algorithm may be used to find a median value in which to use as a reference voltage. When an optimal reference value is established, the highest gain increase can be found without signal over-shoot, therefore improving the dynamic range.

In one example, to preserve the DC information, a DC offset estimate function is used. An analog-to-digital converter (ADC) records the signal after pre-amplification and low-pass filtering of 30 Hz. Utilizing LabView for data acquisition and signal processing, an initial DC offset estimate is acquired and sent to the DAC. This DC offset estimate is used as a reference voltage level for a differential amplifier. Taking the original signal and the DC offset estimate in differential amplification allows the small signal to be extracted with amplification for acquisition by a second ADC to maximize the dynamic range of the system.

To compensate for changing conditions, a reacquisition of the DC offset estimate may be necessary. In order to maximize the resolution and signal-to-noise ratio, input clip detection and signal median estimates are utilized. Sudden changes the subject's position or in the environment, will cause large changes in the DC information. These changes may cause the output signal from the difference amplifier to exceed the range of the small signal ADC. Therefore, a new DC offset estimate will need to be reacquired. Comparators, either as a circuit or within LabView, produce a digital flag to acquire a new value for the DAC. Another condition for a DC offset estimate reacquire flag is from small changes in the nominal distance to the receiver. In order to optimize the signal for maximum dynamic range, the DC offset estimate should be at the median value of the signal. A buffer time set by the user (normally about 4 second) is a periodic call to reacquire the DC offset estimate in conditions of no clip detection. At the end of the buffer time, the dynamic buffer is analyzed and the median value over the buffer period is released to the DAC.

It is noted that the exmplary methods described here can be simplified where the actual value of the DC component is not required or desired for subsequent processing, and can simply be estimated at nominal time increments, and subtracted. Further, it will be recognized that the exemplary DAQ system is illustrative of one possible implementation and that various other implementations are possible and contemplated. For example, various other arrangements and selection of individual components may vary depending on particular applications, cost issues, and the like.

Detection of Multiple Subjects using Generalized Likelihood Ratio Test Methods

According to another aspect of the present invention, a hypothesis test (such as a generalized likelihood ratio test (GLRT)) is described for use in a Doppler radar system. Exemplary GLRT methods and systems may be used for detecting a number (e.g., 0, 1, 2, . . . ) of subjects modulating a transmitted Doppler single for a single transmitter-receiver, SIMO, or MIMO radar sensing system. In one particular example, a GLRT method is based on a model of the heartbeat, and can distinguish between the presence of 0, 1, or 2 subjects (with one or more antennas). Additionally, exemplary GLRT methods and systems described may be extended to N antennas, with detection of up to 2N−1 subjects possible. For example, in a multiple antenna system (SIMO or MIMO), even if individual cardiovascular signatures are very similar, it is possible to distinguish different subjects based on angle or direction of arrival (DOA).

In one example, a continuous wave (CW) radar system transmits a single tone signal at frequency. The model (2) describes the received signal; in particular, the source signal is exp(jKxₛ(t)), where xₛ(t) is the heartbeat and respiration signal. If the wavelength λ is large compared to the maximum displacement of xₛ(t) (which is the case at frequencies below approximately 10 GHz), the complex exponential can be approximated by
exp(jKxₛ(t))≈(1+jKxₛ(t))

The resulting model is therefore (ignoring a DC offset that does not contain information)

$$r ( t ) = {\sum }_{s = 1}^{S} {x}_{s} ( t ) {s}_{s} + w ( t )$$

Here sₛ is a DOA vector (assuming no multipath) that includes various scalar constants.

The signal xₛ(t) generated by a subject typically consists of respiration and heartbeat. The respiration is usually in the range 0-0.8 Hz and the heartbeat in the range 0.8-2 Hz. While the respiration is a stronger signal than the heartbeat, it is also more difficult to characterize and therefore to detect. In this example, most of the respiration may be removed by high pass filtering. The heartbeat signal itself is a rather complicated signal, and although approximately periodic, the period can vary from one beat to the next; this is conventionally referred to as heart rate variability (HRV). HRV can be modeled as a random process with strong periodicity.

In one exemplary GLRT method, and for an instance a single receiver system, with only the I-component available and two subjects in range, the data received in an interval may be modeled as a mixture of two periodic signals:
y[k]=A₁ cos(ω₁kT)+B₁ sin(ω₁kT)+A₂ cos(ω₂kT)+B₂ sin(ω₂kT)+n[k]

where n[k] is white Gaussian noise (WGN) with power σ², and A₁, A₂, B₁, B₂, ω₁, ω₂, and σ² are unknown. It is noted that since n[k] includes terms due to HRV, assuming n[k] is WGN is a rough approximation in the absence of detailed information regarding HRV terms. The problem of determining if there are two or more or less than two persons present can then be stated as
H₁:(A₁,B₁)≠(0,0),(A₂,B₂)≠(0,0)
H₀: (A₂,B₂)=(0,0)

This can be considered a composite hypothesis test problem with many unknown parameters. In one example provided herein, a detector for the above test includes the GLRT. In the GLRT the following test statistic can be defined

$$t ( y ) = \frac{{max}_{{A}_{1} , {B}_{1} , {A}_{2} , {B}_{2} , {ω}_{1} , {ω}_{2} , {σ}^{2}} f ( y )}{{max}_{{A}_{1} , {B}_{1} , {A}_{2} = 0 , {B}_{2} = 0 , {ω}_{1} , {ω}_{2} , {σ}^{2}} f ( y )} \qquad ( 21 )$$

where j(y) is the likelihood function (probability density function) for the received data y=[y[1], . . . , y[N]]. If t(y)>τ, where τ is a threshold, the GLRT decides H₁ (two or more persons), otherwise H₀ (less than two persons). The threshold τ is determined so that a desired false alarm probability is guaranteed. If H₀ is decided, another GLRT can then be used to decide between 0 or 1 subjects.

In the Gaussian case, the GLRT test statistic can be simplified to

$$t ( y ) = \frac{{min}_{{A}_{1} , {B}_{1} , {ω}_{1}} {\sum }_{k = 1}^{N} {( y [ k ] - {A}_{1} cos ( {ω}_{1} t ) - {B}_{1} sin ( {ω}_{1} t ) )}^{2}}{{min}_{{A}_{1} , {B}_{1} , {A}_{2} , {B}_{2} , {ω}_{1} , {ω}_{2}} {\sum }_{k = 1}^{N} {( y [ k ] - {\sum }_{i = 1}^{2} {A}_{i} cos ( {ω}_{i} , t ) + {B}_{i} sin ( {ω}_{i} , t ) )}^{2}}$$

The minimization over A₁, A₂, B₁, B₂ is a linear problem, but the minimization over ω₁, ω₂ is a non-linear problem, which is currently solved using a simple grid search.

The exemplary GLRT methods may be similarly employed with multiple receivers. In other examples where there are multiple receiver antennas (whether SIMO or MIMO systems) with both I and Q-components, and the multipath is negligible, the received signal can be modeled by
y[k]=(A₁ cos(ω₁kT)+B₁ sin(ω₁kT))s(φ₁)+(A₂ cos(ω₂kT)+B₂ sin(ω₂kT))s(φ₂)+n[k]

where s(φ) is given by (21). The GLRT test statistic is now

$$t ( y ) = \frac{min {\sum }_{k = 1}^{N} {‖ ( y [ k ] - {A}_{1} cos ( {ω}_{1} t ) - {B}_{1} sin ( {ω}_{1} t ) ) s ( {ϕ}_{1} ) ‖}^{2}}{min {{\sum }_{k = 1}}^{N} {‖ y [ k ] - {\sum }_{i = 1}^{2} ( {A}_{i} cos ( {ω}_{i} t ) - {B}_{i} sin ( {ω}_{i} t ) ) s ( {ϕ}_{i} ) ‖}^{2}} \qquad ( 22 )$$

Now the minimization ω₁, ω₂, φ₁, φ₂ is a non-linear problem solved using a simple grid search. Notice that the minimization with respect to φ₁, φ₂ gives DOA as a by-product, so the methods can also be used to localizing subjects.

An exemplary apparatus includes a single transmitter-receiver system similar to that illustrated FIG. 1. The apparatus may include a CW signal source at 2.4 GHz with 0 dBm output power. The transmitted signal, having been modulated by a subject, is mixed with a sample of the transmitted signal to produce an output voltage with its magnitude proportional to the phase shift between them, which in turn is proportional to chest displacement due to cardiopulmonary activity.

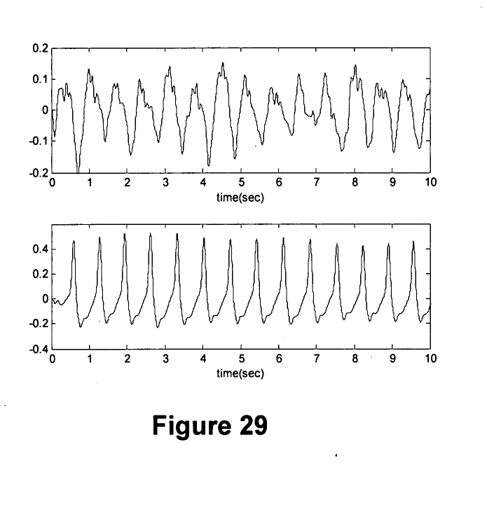

FIG. 29 illustrates a heart beat signal from a subject as measured by a transmitter-receiver system as described, and compared with a reference signal of the subject from a finger sensor. The sensed signal is filtered with a lowpass filter with cutoff 10 Hz. It can be seen that the sensed signal, compared with the reference signal, is relatively noisy, and a heartbeat rate cannot be easily determined from simple peak detection.

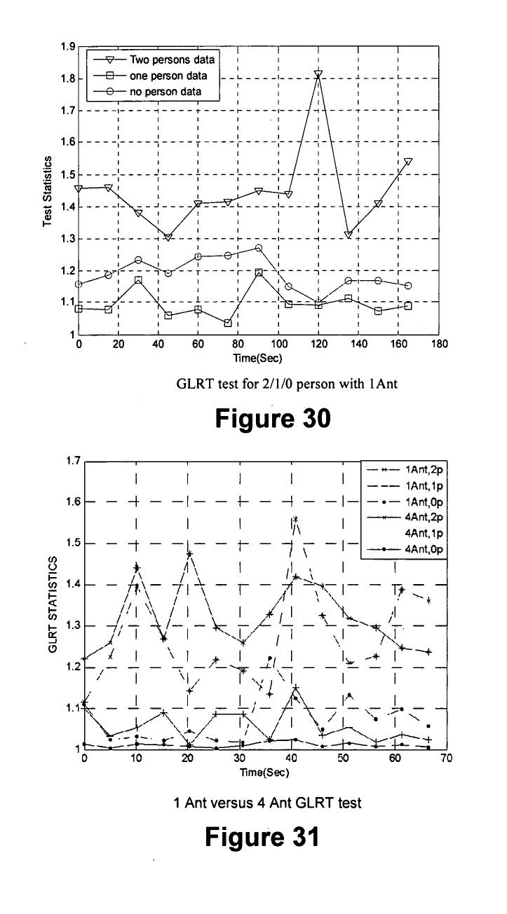

FIG. 30 illustrates a plot of test statistic (22) applied to three different sets of measurements with a single antenna; in particular, testing for the presence of 0, 1, or 2 subjects. The measurements are first bandpass filtered with a passband 0.8-2 Hz to remove respiration and higher order harmonics, and are then divided into (overlapping) intervals of length 15s (to ensures that the model is reasonably accurate). The test statistic is now evaluated in each 15s interval and determines the number of subjects within range, e.g., by using threshold of 1.25. Note that once the exemplary method and system determines that less than two subjects are present, another exemplary GLRT can be applied to distinguish 0 and 1 subjects.

FIG. 31 illustrates partial simulation data for comparing distinguishing 1 subject from 2 subjects. In this example, reference signals were measured for different subjects, multiplied with DOA vectors, and independent noise added at each antenna. In this case a subject with strong HRV was used for reference data. As shown, with the exemplary set-up, a single antenna system did not reliable detect if there are 1 or 2 subjects. With multiple antennas, in this instance with 4 antennas, the system reliably distinguished between 1 and 2 subjects within range.

In another example, a singular value decomposition (SVD) combination may be used to combine channel data to extract physiological motion (e.g., heartbeat signals). The resulting signal may include the principle component of heartbeat signal, with maximal output SNR among all I and Q channels. For GLRT methods, the MLE of unknown parameters is solved first, and in one example, a method and system is based on FFT and GLRT, referred to herein as a FFT-GLRT-based detector.

First, an exemplary method is described when noise is unknown, followed by an exemplary method when noise is known, and for complex systems where DOA and distance of subjects are used. Assuming the data received is real, detection frame by frame with length of MN is performed. Each frame can be divided into M subwindows, which contains N samples. The measurement can be written as
xₘ[n]=sₘ[n]+wₘ[n]

where Xₘ[n] is the received signal. ωₘ[n] is a sequence of independent, identically distributed zero mean Gaussian noise with unknown variance σ². Assuming a start sample at t=0. If not, the initial phase can be combined into θₘ, then
sₘ[n]=Aₘ cos(wtₘₙ+θₘ)=aₘ cos(wtₘₙ)+bₘ sin(wtₘₙ)
tₘₙ=(m·N+n)T

The joint density function for the random sample x=(x₀, x₁, . . . , x_{MN-1}) is the product density

$${f}_{Θ} ( x ; {H}_{1} ) = {( 2 {πσ}^{2} )}^{- MN / 2} exp - \{ \frac{1}{2 {σ}^{2}} {\sum }_{m = 0}^{M - 1} {\sum }_{n = 0}^{N - 1} [ {( {x}_{m} [ n ] - {s}_{m} [ n ] )}^{2} ] \}$$

$$Θ = [ ω , \overline{a} , \overline{b} , {σ}^{2} ]$$

Initially, find the maximum likelihood estimate of Θ, in particular the frequency ω. To maximize the log-likelihood L_{Θ}(x; H₁)=1n f_{Θ}(x) first with respect σ²:

$$\frac{\partial }{\partial {σ}^{ 2}} {L}_{ Θ} ( x ; {H}_{ 1} ) = - \frac{MN}{ 2 {σ}^{ 2}} + \frac{1}{ 2 {σ}^{ 4}} {\sum }_{m = 0}^{ M - 1} {\sum }_{n = 0}^{ N - 1} {( {x}_{ m} [ n ] - {s}_{ m} [ n ] )}^{2} = 0$$

Define the square error γ²ₘ and γ²: as

$${γ}_{m}^{2} = {\sum }_{n = 0}^{N - 1} {( {x}_{m} [ n ] - {s}_{m} [ n ] )}^{2}$$

$${γ}^{2} = {\sum }_{m = 0}^{M - 1} {\sum }_{n = 0}^{N - 1} {( {x}_{m} [ n ] - {s}_{m} [ n ] )}^{2} = {\sum }_{m = 0}^{M - 1} {γ}_{m}^{2}$$

So the maximum likelihood estimate of 2 is

$${\hat{σ}}^{2} = \frac{1}{MN} {γ}^{2} = \frac{1}{MN} {\sum }_{m = 0}^{M - 1} {\sum }_{n = 0}^{N - 1} {( {x}_{m} [ n ] - {s}_{m} [ n ] )}^{2}$$

Thus, we have the likelihood and log-likelihood as

$${f}_{Θ} ( x ; {H}_{1} ) = {( 2 π {\hat{σ}}^{2} )}^{- \frac{MN}{2}} exp \{ - \frac{MN}{2} \}$$

$${L}_{Θ} ( x ; {H}_{1} ) = - \frac{MN}{2} ln ( 2 π {\hat{σ}}^{2} ) - \frac{MN}{2}$$

To maximize the log-likelihood L_{Θ}(x; H₁) is equivalent to minimize the square error γ², the summation of γ²ₘ over m_{m. Since γ}². Each

$${item}_{m}$$

of γ²ₘ contains unknown parameters ω, aₘ, bₘ. To determine them, first expand ₘγ²ₘ :

$$\begin{matrix}{\sum }_{n = 0}^{ N - 1} [ {( {x}_{ m} [ n ] - {s}_{ m} [ n ] )}^{2} ] = {\sum }_{n = 0}^{ N - 1} {[ {x}_{ m} [ n ] - {a}_{ m} cos ( ω {t}_{ mn} ) - {b}_{ m} sin ( ω {t}_{ mn} ) ]}^{2} \\ = {\sum }_{n = 0}^{N - 1} {{x}_{m} [ n ]}^{2} + {a}_{m} {b}_{m} {\sum }_{n = 0}^{N - 1} sin ( 2 ω {t}_{mn} ) + \\ {a}_{m}^{2} {\sum }_{n = 0}^{N - 1} {cos}^{2} ( ω {t}_{mn} ) + {b}_{m}^{2} {\sum }_{n = 0}^{N - 1} {sin}^{2} ( ω {t}_{mn} ) - \\ {a}_{m} [ {\sum }_{n = 0}^{N - 1} {x}_{m} [ n ] ( {ⅇ}^{- j n ω T} {ⅇ}^{- j mN ω T} + {ⅇ}^{j n ω T} {ⅇ}^{j mN ω T} ) ] + \\ {jb}_{m} [ {\sum }_{n = 0}^{N - 1} {x}_{m} [ n ] ( {ⅇ}^{- j n ω T} {ⅇ}^{- j mN ω T} - {ⅇ}^{j n ω T} {ⅇ}^{j mN ω T} ) ]\end{matrix}$$

From the definition of Discrete Fourier Transform (DFT),

$${X}_{m} = ( ω ) = {\sum }_{n = 0}^{N - 1} {x}_{m} [ n ] {ⅇ}^{- j n ω T}$$

in connection with approximation

$${\sum }_{n = 0}^{N - 1} {cos ( ω {t}_{mn} )}^{2} \approx \frac{N}{2}$$

$${\sum }_{n = 0}^{N - 1} {sin ( ω {t}_{mn} )}^{2} \approx \frac{N}{2}$$

$${\sum }_{n = 0}^{N - 1} sin ( 2 ω {t}_{mn} ) \approx 0$$

which leads to

$${γ}_{m}^{2} = {\sum }_{n = 0}^{N - 1} {{x}_{m} [ n ]}^{2} + \frac{N}{2} ( {a}_{m}^{2} + {b}_{m}^{2} ) - \\ {a}_{m} [ {X}_{m} ( ω ) {ⅇ}^{- j mN ω T} + {X}_{m} ( - ω ) {ⅇ}^{j m N ω T} ] + \\ {jb}_{m} [ {X}_{m} ( ω ) {ⅇ}^{- j m N ω T} - {X}_{m} ( - ω ) {ⅇ}^{j m N ω T} ]$$

Because Xₘ is real,
Xₘ(−w)e^{jmNut}={Xₘ(w)e−^{jmNwT}}

Then

$${γ}_{m}^{2} = {\sum }_{n = 0}^{N - 1} {{x}_{m} [ n ]}^{2} + \frac{N}{2} ( {a}_{m}^{2} + {b}_{m}^{2} ) - 2 {a}_{m} Re [ {X}_{m} ( ω ) {ⅇ}^{- j m N ω T} ] + 2 {b}_{m} Im [ {X}_{m} ( ω ) {ⅇ}^{- j m N ω T} ]$$

As assumed, phase jumps and magnitude changes from subwindow to subwindow, then the parameters aₘ, bₘ are independent with each other. Take first derivative to get solutions of aₘ, bₘ that make the square error γ² minimal,

$$\frac{\partial {γ}^{2}}{\partial {a}_{m}} = \frac{\partial {γ}_{m}^{2}}{\partial {a}_{m}} = N {a}_{m} - 2 Re [ {X}_{m} ( ω ) {ⅇ}^{- j m N ω T} ]$$

By taking derivative with bm leads to the solutions:

$${\hat{a}}_{m} = \frac{2}{N} Re [ {X}_{m} ( ω ) {ⅇ}^{- j m N ω T} ]$$

$${\hat{b}}_{m} = - \frac{2}{N} Im [ {X}_{m} ( ω ) {ⅇ}^{- j m N ω T} ]$$

And γ² can be given as

$$\begin{matrix}{γ}_{m}^{2} = {\sum }_{n = 0}^{N - 1} {x}_{m}^{2} [ n ] - \frac{N}{2} ( {\hat{a}}_{m}^{2} + {\hat{b}}_{m}^{2} ) \\ = {\sum }_{n = 0}^{N - 1} {x}_{m}^{2} [ n ] - \frac{2}{N} {‖ {X}_{m} ( ω ) {ⅇ}^{ - j m N ω T} ‖}^{2} \\ = {\sum }_{n = 0}^{N - 1} {x}_{m}^{2} [ n ] - \frac{2}{N} {‖ {X}_{m} ( ω ) ‖}^{2}\end{matrix}$$

Hence, to maximize the log-likelihood L_{Θ}(x; H₁) is equivalent to minimizing

$${γ}^{2} = {\sum }_{m = 0}^{M - 1} {\sum }_{n = 0}^{N - 1} {x}_{m}^{2} [ n ] - \frac{2}{N} {\sum }_{m = 0}^{M - 1} {‖ {X}_{m} ( ω ) ‖}^{2}$$

For fixed M and N, the value of γ² only depends on w now, thus the maximum likelihood estimate of ω:

$${\hat{ω}}_{ML} = {max}_{ω} \frac{1}{M} {\sum }_{m = 0}^{M - 1} {‖ {X}_{m} ( ω ) ‖}^{2}$$

Finally, summarizing all the parameter estimations under H₁ hypothesis

$$\begin{matrix}{\hat{ω}}_{ML} = {max}_{ω} \frac{1}{M} {\sum }_{m = 0}^{M - 1} {‖ {X}_{m} ( ω ) ‖}^{2} \\ {\hat{a}}_{m} = \frac{2}{N} Re \{ {X}_{m} ( \hat{ω} ) {ⅇ}^{- j m N \hat{ω} T} \} \\ {\hat{b}}_{m} = - \frac{2}{N} Im \{ {X}_{m} ( ω ) {ⅇ}^{- j m N \hat{ω} T} \} \\ {\hat{σ}}_{1}^{2} = \frac{1}{MN} \{ {\sum }_{m = 0}^{M - 1} {\sum }_{n = 0}^{N - 1} {x}_{m}^{2} [ n ] - \frac{2}{N} {\sum }_{m = 0}^{M - 1} {‖ {X}_{m} ( \hat{ω} ) ‖}^{2} \} \\ {f}_{⊖} ( x ; {H}_{1} ) = {( 2 π {\hat{σ}}_{1}^{2} )}^{- \frac{MN}{2}} exp ( - \frac{MN}{2} )\end{matrix}$$

Under H₀ hypothesis, one only need to estimate the noise variance

$$\begin{matrix}\hat{{σ}_{0}^{2}} = \frac{1}{M N} {\sum }_{m = 0}^{M - 1} {\sum }_{n = 0}^{N - 1} {x}_{m}^{2} [ n ] \\ {f}_{Θ} ( x ; {H}_{0} ) = {( 2 π \hat{{σ}_{0}^{2}} )}^{- \frac{M N}{2}} exp ( - \frac{M N}{2} )\end{matrix}$$

Now the likelihood ratio for hypothesis H₁ and H₀ can be represented as

$${L}_{G} ( x ) = \frac{{f}_{Θ} ( x ; {H}_{1} )}{{f}_{Θ} ( x ; {H}_{0} )} = {( \frac{{σ}_{1}^{2}}{\hat{{σ}_{0}^{2}}} )}^{- \frac{M N}{2}}$$

Since M and N are known, the test statistics can be expressed as

$$\begin{matrix}{t}_{G} ( x ) = \frac{{\sum }_{m = 0}^{M - 1} {\sum }_{n = 0}^{N - 1} {x}_{m}^{2} [ n ]}{{\sum }_{m = 0}^{M - 1} {\sum }_{n = 0}^{N - 1} {[ {x}_{m} [ n ] - {\hat{a}}_{m} cos ( \hat{ω} {t}_{mn} ) - {\hat{b}}_{m} sin ( \hat{ω} {t}_{mn} ) ]}^{2}} \\ = \frac{{\sum }_{m = 0}^{M - 1} {\sum }_{n = 0}^{N - 1} {x}_{m}^{2} [ n ]}{{\sum }_{m = 0}^{M - 1} {\sum }_{n = 0}^{N - 1} {x}_{m}^{2} [ n ] - \frac{2}{N} {\sum }_{m = 0}^{M - 1} {‖ {X}_{m } ( {\hat{ω}}_{M L} ) ‖}^{2}}\end{matrix} \qquad ( 6.1 .26 )$$

To get test statistics, one can evaluate the power of a received signal, and search the peak of averaged PSD. With respect to the narrow range of heartbeat frequency (e.g., 0.8˜2 Hz), the processing speed may be increased with use of a Goertzel algorithm instead of a classical FFT.

In the case of known noise, the joint density function for the random process of ω(Θ, t) is almost the same, and the only difference is that the random vector Θ doesn't include σ² any more

$$\begin{matrix}{f}_{Θ} ( x ; {H}_{1} ) = {( 2 {πσ}^{2} )}^{- M N / 2} exp \{ - \frac{1}{2 {σ}^{2}} {\sum }_{m = 0}^{M - 1} {\sum }_{n = 0}^{N - 1} [ {( {x}_{m} [ n ] - {s}_{m} [ n ] )}^{2} ] \} \\ {f}_{Θ} ( x ; {H}_{0} ) = {( 2 {πσ}^{2} )}^{- M N / 2} exp \{ - \frac{1}{2 {σ}^{2}} {\sum }_{m = 0}^{M - 1} {\sum }_{n = 0}^{N - 1} {x}_{m}^{2} [ n ] \} \\ Θ = [ ω , \overline{a} , b ]\end{matrix}$$

The likelihood ratio for hypothesis H₁ and H₀ can be denoted as

$${L}_{G} ( x ) = \frac{exp \{ \frac{1}{2 {σ}^{2}} {\sum }_{m = 0}^{M - 1} {\sum }_{n = 0}^{N - 1} {x}_{m}^{2} [ n ] \}}{exp \{ \frac{1}{2 {σ}^{2}} {\sum }_{m = 0}^{M - 1} {\sum }_{n = 0}^{N - 1} {[ {x}_{m} [ n ] - {\hat{a}}_{m} cos ( \hat{ω} {t}_{mn} ) - {\hat{b}}_{m} sin ( \hat{ω} {t}_{mn} ) ]}^{2} \}}$$

When M and N are known, after talung logarithm of the likelihood ratio, the test statistics is expressed as

$$\begin{matrix}{t}_{G} ( x ) = \frac{1}{2 {σ}^{2}} \{ {\sum }_{m = 0}^{M - 1} {\sum }_{n = 0}^{N - 1} {x}_{m}^{2} [ n ] - {\sum }_{m = 0}^{M - 1} {\sum }_{n = 0}^{N - 1} {[ \begin{matrix}{x}_{m} [ n ] - {\hat{a}}_{m} cos ( \hat{ω} {t}_{mn} ) - \\ {\hat{b}}_{m} sin ( \hat{ω} {t}_{mn} )\end{matrix} ]}^{2} \} \\ = \frac{1}{2 {σ}^{2}} \{ {\sum }_{m = 0}^{M - 1} {\sum }_{n = 0}^{N - 1} {x}_{m}^{2} [ n ] - ( {\sum }_{m = 0}^{M - 1} {\sum }_{n = 0}^{N - 1} {x}_{m}^{2} [ n ] - \frac{2}{N} {\sum }_{m = 0}^{M - 1} {‖ {X}_{m} ( \hat{ω} ) ‖}^{2} ) \} \\ = \frac{1}{N {σ}^{2}} {\sum }_{m = 0}^{M - 1} {‖ {X}_{m} ( {\hat{ω}}_{ML} ) ‖}^{2}\end{matrix}$$

Additionally, detection for complex data model, e.g., includes DOA and the distance of each subject, will now be discussed. In SVD combination, the characteristics of DOA and distance of each subject was not fully exploited. However, these characteristics are beneficial to identify subjects, especially when multiple subjects are present. Although the SVD combined data can provide a higher accuracy for frequency estimation, it does not assure to result in improved detection performance. If the IQ measurement is correct, the complex data should perform better in detection, because it contains more information than SVD combined data. We will investigate how to use data of both IQ channels more efficiently, and evaluate its detection performance.

For each window, assume the mth subwindow, nth sample can be given by:
zₘ[n]=sₘ[n]+wₘ[n]=xₘ[n]+jyₘ[n]

where (xₘ[n], yₘ [n]) is the received I and Q data; wₘ[n] is a sequence of independent, identically distributed zero mean complex Gaussian noise with unknown variance σ². Assume Aₘ and Bₘ are the magnitudes for I and Q:
Aₘ=−A sin(φ)w_{c}=C cos(Ψ)
Bₘ=A cos(ω)w_{c}=C sin(Ψ)

C is constant so it can be combined into aₘ and bₘ. Ψ is introduced for simplification, which is also constant within a detection window. Then the IQ data can also be given by

$$\begin{matrix}{x}_{m} [ n ] = {A}_{m} cos ( ω {t}_{mn} + {θ}_{m} ) + Re \{ {w}_{m} [ n ] \} \\ = cos ( ψ ) [ {a}_{m} cos ( {ωt}_{mn} ) + {b}_{m} sin ( {ωt}_{mn} ) ] + Re \{ {w}_{m} [ n ] \} \\ {y}_{m} [ n ] = {B}_{m} cos ( ω {t}_{mn} + {θ}_{m} ) + Im \{ {w}_{m} [ n ] \} \\ = sin ( ψ ) [ {a}_{m} cos ( ω {t}_{mn} ) + {b}_{m} sin ( ω {t}_{mn} ) ] + Im \{ {w}_{m} [ n ] \} \\ {t}_{mn} = ( m \cdot N + n ) T\end{matrix}$$

The joint density function for the random sample z=(z₀, z₁ . . . , z_{MN-1}) is

$$\begin{matrix}{f}_{Θ} ( z ; {H}_{1} ) = {( 2 π {σ}^{2} )}^{- MN} exp \{ - \frac{1}{2 {σ}^{2}} {\sum }_{m = 0}^{M - 1} {\sum }_{n = 0}^{N - 1} {‖ {z}_{m} [ n ] - {s}_{m} [ n ] ‖}^{2} \} \\ Θ = [ ω , \overline{a} , \overline{b} , ψ , {σ}^{2} ]\end{matrix}$$

The magnitude and DOA are independent of the heartbeat's frequency and phase. Hence, Ψ is independent of ω, aₘ, bₘ. The γ²=Σₘ₌₀^{M-1}γₘ² and γₘ²

$${γ}_{m}^{2} = {\sum }_{n = 0}^{N - 1} {{x}_{m} [ n ]}^{2} + {\sum }_{n = 0}^{N - 1} {{y}_{m} [ n ]}^{2} + \{ {a}_{m} {b}_{m} {\sum }_{n = 0}^{N - 1} sin ( 2 ω {t}_{mn} ) + {a}_{m}^{2} {\sum }_{n = 0}^{N - 1} {cos}^{2} ( ω {t}_{mn} ) + {b}_{m}^{2} {\sum }_{n = 0}^{N - 1} {sin}^{2} ( ω {t}_{mn} ) \} - cos ( ψ ) {a}_{m} [ {\sum }_{n = 0}^{N - 1} {x}_{m} [ n ] ( {ⅇ}^{- j n ω T} {ⅇ}^{- j ( k M + m ) N ω T} + {ⅇ}^{j n ω T} {ⅇ}^{j ( k M + m ) N ω T} ) ] - jcos ( ψ ) {b}_{m} [ {\sum }_{n = 0}^{N - 1} {x}_{m} [ n ] ( {ⅇ}^{- j n ω T} {ⅇ}^{- j ( k M + m ) N ω T} - {ⅇ}^{j n ω T} {ⅇ}^{j ( k M + m ) N ω T} ) ] - sin ( ψ ) {a}_{m} [ {\sum }_{n = 0}^{N - 1} {y}_{m} [ n ] ( {ⅇ}^{- j n ω T} {ⅇ}^{- j ( k M + m ) N ω T} + {ⅇ}^{j n ω T} {ⅇ}^{j ( k M + m ) N ω T} ) ] - jsin ( ψ ) {b}_{m} [ {\sum }_{n = 0}^{N - 1} {y}_{m} [ n ] ( {ⅇ}^{- j n ω T} {ⅇ}^{- j ( k M + m ) N ω T} - {ⅇ}^{j n ω T} {ⅇ}^{j ( k M + m ) N ω T} ) ]$$

By the definition of Discrete Fourier Transform (DFT),

$$\begin{matrix}{X}_{m} ( ω ) = {\sum }_{n = 0}^{N - 1} {x}_{m} [ n ] {ⅇ}^{- j n ω T} \\ {Y}_{m} ( ω ) = {\sum }_{n = 0}^{N - 1} {y}_{m} [ n ] {ⅇ}^{- j n ω T}\end{matrix}$$

and properties of real data's DFT
Xₘ(−w)e^{j(kM+m)NwT}={Xₘ(w)e^{-j(kM+m)NwT}}*
Yₘ(−w)e^{j(kM+m)NwT}={Yₘ(w)e^{-i(kM+m)NwT}}*

and simplified denotation
\tilde{X}ₘ(w)=Xₘ(w)e^{-j(kM+m)NwT}, \tilde{Y}ₘ(w)=Yₘ(w)e^{-j(kM+m)NwT}
in connection with approximation

$$\begin{matrix}{\sum }_{n = 0}^{N - 1} {cos}^{2} ( ω {t}_{mn} ) \approx \frac{N}{2} \\ {\sum }_{n = 0}^{N - 1} {sin}^{2} ( ω {t}_{mn} ) \approx \frac{N}{2} \\ {\sum }_{n = 0}^{N - 1} sin ( 2 ω {t}_{mn} ) \approx 0\end{matrix}$$

Where γ² can be simplified as

$${γ}_{m}^{2} = {\sum }_{n = 0}^{N - 1} {{x}_{m} [ n ]}^{2} + {\sum }_{n = 0}^{N - 1} {{y}_{m} [ n ]}^{2} + \frac{N}{2} ( {a}_{m}^{2} + {b}_{m}^{2} ) - cos ( ψ ) {a}_{m} \cdot 2 Re [ {\tilde{X}}_{m} ( ω ) ] + cos ( ψ ) {b}_{m} \cdot 2 Im [ {\tilde{X}}_{m} ( ω ) ] - sin ( ψ ) {a}_{m} \cdot 2 Re [ {\tilde{Y}}_{m} ( ω ) ] + sin ( ψ ) {b}_{m} \cdot 2 Im [ {\tilde{Y}}_{m} ( ω ) ]$$

To get solutions that make the square error γ² minimal, one can take first derivative with aₘ, bₘ, Ψ separately. Since aₘ, bₘ are independent with each other, then

$$\begin{matrix}\begin{matrix}\frac{\partial {γ}^{2}}{\partial {a}_{m}} = \frac{\partial {γ}_{m}^{2}}{\partial {a}_{m}} = N {a}_{m} - 2 cos ( ψ ) Re [ {\tilde{X}}_{m} ( ω ) ] - sin ( ψ ) Re [ {\tilde{Y}}_{m} ( ω ) ] = 0 \\ \frac{\partial {γ}^{2}}{\partial {b}_{m}} = \frac{\partial {γ}_{m}^{2}}{\partial {b}_{m}} = N {b}_{m} + 2 cos ( ψ ) Im [ {\tilde{X}}_{m} ( ω ) ] + 2 sin ( ψ ) Im [ {\tilde{Y}}_{m} ( ω ) ] = 0\end{matrix} & ( 23 ) \\ \begin{matrix}\frac{\partial {γ}^{2}}{\partial ψ} = {\sum }_{m = 0}^{M - 1} \frac{\partial {γ}_{m}^{2}}{\partial ψ} \\ \frac{\partial {γ}_{m}^{2}}{\partial ψ} = sin ( ψ ) \{ {a}_{m} \cdot 2 Re [ {\tilde{X}}_{m} ( ω ) ] - {b}_{m} \cdot 2 Im [ {\tilde{X}}_{m} ( ω ) ] \} - \\ cos ( ψ ) \{ {a}_{m} \cdot 2 Re [ {\tilde{Y}}_{m} ( ω ) ] - {b}_{m} \cdot 2 Im [ {\tilde{Y}}_{m} ( ω ) ] \}\end{matrix} & ( 24 )\end{matrix}$$

From the equation (23) above, one can get

$$\begin{matrix}{\hat{a}}_{m} = \frac{2}{N} \{ cos ( ψ ) Re [ {\tilde{X}}_{m} ( ω ) ] + sin ( ψ ) Re [ {\tilde{Y}}_{m} ( ω ) ] \} \\ {\hat{b}}_{m} = - \frac{2}{N} \{ cos ( ψ ) Im [ {\tilde{X}}_{m} ( ω ) ] + sin ( ψ ) Im [ {\tilde{Y}}_{m} ( ω ) ] \}\end{matrix}$$

Substitute the above result into (24), and with
Re[\tilde{X}ₘ(w)]Re[\tilde{Y}ₘ(w)]+Im[\tilde{X}ₘ(w)]Im[\tilde{Y}ₘ(w)]=Re[\tilde{X}ₘ(w)Yₘ*(w)]

One can get

$$\begin{matrix}\frac{\partial {γ}_{m}^{2}}{\partial ψ} = \frac{4}{N} sin ( ψ ) cos ( ψ ) ( {‖ {\tilde{X}}_{m} ( ω ) ‖}^{2} - {‖ {\tilde{Y}}_{m} ( ω ) ‖}^{2} ) - \\ \frac{4}{N} ( {cos}^{2} ( ψ ) - {sin}^{2} ( ψ ) ) Re [ {\tilde{X}}_{m} ( ω ) {\tilde{Y}}_{m}^{*} ( ω ) ] \} \\ Then \\ \frac{\partial {γ}^{2}}{\partial ψ} = \frac{2}{N} sin ( 2 ψ ) {\sum }_{m = 0}^{M - 1} [ {‖ {\tilde{X}}_{m} ( ω ) ‖}^{2} - {‖ {\tilde{Y}}_{m} ( ω ) ‖}^{2} ] - \\ \frac{4}{N} cos ( 2 ψ ) {\sum }_{m = 0}^{M - 1} Re [ {\tilde{X}}_{m} ( ω ) {\tilde{Y}}_{m}^{*} ( ω ) ]\end{matrix}$$

Or express it as (assume Ψ≠(2N+1)π/4)

$$tan ( 2 ψ ) = \frac{2 {\sum }_{m = 0}^{M - 1} Re [ {\tilde{X}}_{m} ( ω ) {\tilde{Y}}_{m}^{*} ( ω ) ]}{{\sum }_{m = 0}^{M - 1} [ {‖ {\tilde{X}}_{m} ( ω ) ‖}^{2} - {‖ {\tilde{Y}}_{m} ( ω ) ‖}^{2} ]} \qquad ( 6.2 .18 )$$

Then the solution is

$$ψ = \frac{1}{2 } arctan \{ \frac{2 {\sum }_{m = 0}^{M - 1} Re [ {\tilde{X}}_{m} ( ω ) {\tilde{Y}}_{m}^{*} ( ω ) ]}{{\sum }_{m = 0}^{M - 1} [ {‖ {\tilde{X}}_{m} ( ω ) ‖}^{2} - {‖ {\tilde{Y}}_{m} ( ω ) ‖}^{2} ]} \} \qquad ( 6.2 .19 )$$

For square error

$$\begin{matrix}{γ}_{m}^{2} = {\sum }_{n = 0}^{N - 1} {{x}_{m} [ n ]}^{2} + {\sum }_{n = 0}^{N - 1} {[ n ]}^{2} - \frac{N}{2} ( {\hat{a}}_{m}^{2} + {\hat{b}}_{m}^{2} ) \\ = {\sum }_{n = 0}^{N - 1} {{x}_{m} [ n ]}^{2} + {\sum }_{n = 0}^{N - 1} {{y}_{m} [ n ]}^{2} - \frac{2}{N} \{ {cos}^{2} ( ψ ) {‖ {\tilde{X}}_{m} ( ω ) ‖}^{2} + {sin}^{2} ( ψ ) {‖ {\tilde{Y}}_{m} ( ω ) ‖}^{2} + \\ sin ( 2 ψ ) Re [ {\tilde{X}}_{m} ( ω ) {\tilde{Y}}_{m}^{*} ( ω ) ] \}\end{matrix}$$

One can see, for each value of ω, there is a corresponding Ψ. For γ², let's define the part contain function of Ψ as βₘ(ω, Ψ). βₘ(ω, Ψ) can simplified as

$$\begin{matrix}{γ}_{m}^{2} = {\sum }_{n = 0}^{N - 1} {{x}_{m} [ n ]}^{2} + {\sum }_{n = 0}^{N - 1} {{y}_{m} [ n ]}^{2} - {β}_{m} ( ω , ψ ) \\ {β}_{m} ( ω , ψ ) = \frac{1}{N} \{ {‖ {\tilde{X}}_{m} ( ω ) ‖}^{2} + {‖ {\tilde{Y}}_{m} ( ω ) ‖}^{2} + sec ( 2 ψ ) [ {‖ {\tilde{X}}_{m} ( ω ) ‖}^{2} - {‖ {\tilde{Y}}_{m} ( ω ) ‖}^{2} ] \}\end{matrix}$$

If Ψ is known, for determinate M and N, the value of γ² only depends on (o, thus the maximum likelihood estimate of ω:

$$\tilde{ω} = {max}_{ω} {\sum }_{m = 0}^{M - 1} {β}_{m} ( ω , ψ )$$

Notice that, the estimation of ω contains Ψ, and estimation of Ψalso contains ω.

Finally, we summarize all the parameter estimations under H₁ hypothesis:

$$\begin{matrix}\hat{ω} = {max}_{ω} {\sum }_{m = 0}^{M - 1} {β}_{m} ( ω , ψ ) \\ {β}_{m} ( ω , ψ ) = \frac{1}{N} \{ \begin{matrix}{‖ {\tilde{X}}_{m} ( ω ) ‖}^{2} + {‖ {\tilde{Y}}_{m} ( ω ) ‖}^{2} + \\ csc ( 2 ψ ) [ {‖ {\tilde{X}}_{m} ( ω ) ‖}^{2} - {‖ {\tilde{Y}}_{m} ( ω ) ‖}^{2} ]\end{matrix} \} \\ \hat{ψ} = \frac{1}{2} arc tan \{ \frac{2 {\sum }_{m = 0}^{M - 1} Re [ {\tilde{X}}_{m} ( ω ) {\tilde{Y}}_{m}^{*} ( ω ) ]}{{\sum }_{m = 0}^{M - 1} [ {‖ {\tilde{X}}_{m} ( ω ) ‖}^{2} - {‖ {\tilde{Y}}_{m} ( ω ) ‖}^{2} ]} \} \\ {\hat{a}}_{m} = \frac{2}{N} \{ cos ( ψ ) Re [ {\tilde{X}}_{m} ( \hat{ω} ) ] + sin ( ψ ) Re [ {\tilde{Y}}_{m} [ \hat{ω} ) ] \} \\ {\hat{b}}_{m} = - \frac{2}{N} \{ cos ( ψ ) Im [ {\tilde{X}}_{m} ( \hat{ω} ) ] + sin ( ψ ) Im [ {\tilde{Y}}_{m} ( \hat{ω} ) ] \} \\ {\hat{σ}}_{1}^{2} = \frac{1}{MN} \{ {\sum }_{m = 0}^{M - 1} {\sum }_{n = 0}^{N - 1} ( {x}_{m}^{2} [ n ] + {y}_{m}^{2} [ n ] ) - {\sum }_{m = 0}^{M - 1} {β}_{m} ( \hat{ω} , \hat{ψ} ) \} \\ {f}_{⊖} ( x , y ; {H}_{1} ) = {( 2 π {\hat{σ}}_{1}^{2} )}^{- MN} exp ( - MN )\end{matrix}$$

Now one can represent the likelihood ratio for hypothesis H₁ and H₀ as

$${L}_{G} ( x , y ) = \frac{{f}_{⊖} ( x , y ; {H}_{1} )}{{f}_{⊖} ( x , y ; {H}_{0} )} = {( \frac{{\hat{σ}}_{1}^{2}}{{\hat{σ}}_{0}^{2}} )}^{- MN}$$

Since M and N are known, for each window, the test statistics can also be represented as

$${t}_{G} ( x , y ) = \frac{{\sum }_{m = 0}^{M - 1} {\sum }_{n = 0}^{N - 1} ( {x}_{m}^{2} [ n ] + {y}_{m}^{2} [ n ] )}{{\sum }_{m = 0}^{M - 1} {\sum }_{n = 0}^{N - 1} ( {x}_{m}^{2} [ n ] + {y}_{m}^{2} [ n ] ) - {\sum }_{m = 0}^{M - 1} {‖ {β}_{m} ( \hat{ω} ) ‖}^{2}}$$

To get test statistics, one only need evaluate the power of the received signal, and search the peak of modified averaged PSD. In one example, one can also substitute Goertzel algorithm with FFT. Although it's more complex to get modified averaged PSD, the detector is still based on GLRT and FFT. Therefore, its computation is not much heavier. On the other hand, the complex data model does not require SVD combination.

Accordingly, exemplary methods and systems are provided for determining the number of subjects within range using hypothesis testing; in particular, a GLRT. The methods and systems may detect up to 2N subjects with N antennas. Various modification to the exemplary method and system are possible. For example, the exemplary method could be simplified by using an approximate minimization, for example by using 2D FFT and peak search.

While aspects of the invention, including the above described methods, are described in terms of particular embodiments and illustrative figures, those of ordinary skill in the art will recognize that the invention is not limited to the embodiments or figures described. Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic. (The term “logic” herein refers to fixed hardware, programmable logic, and/or an appropriate combination thereof, as would be recognized by one skilled in the art to carry out the recited functions.) Software and firmware can be stored on computer-readable media. Some other processes can be implemented using analog circuitry, as is well known to one of ordinary skill in the art. Additionally, memory or other storage, as well as communication components, may be employed in embodiments of the invention.

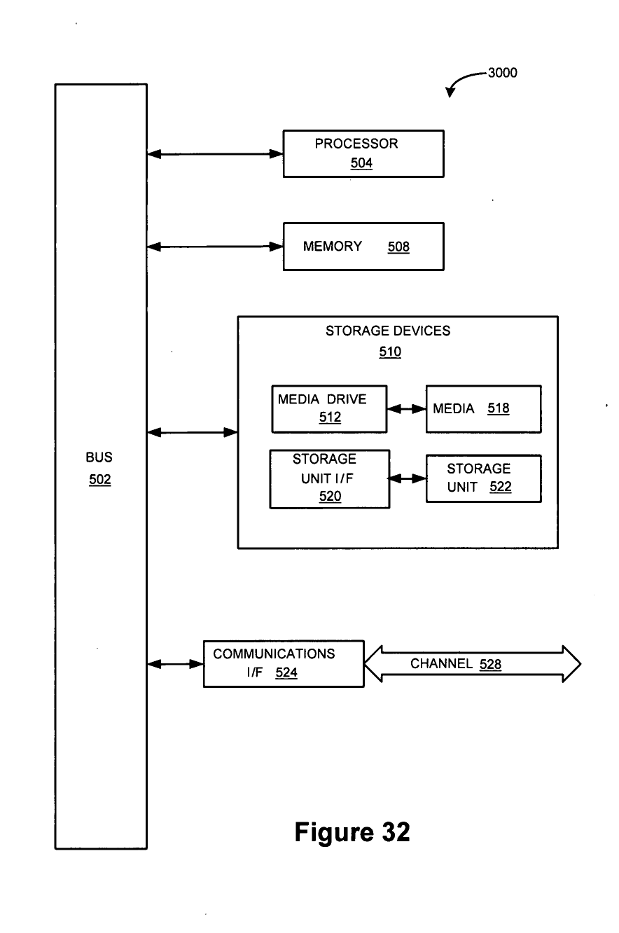

FIG. 32 illustrates an exemplary measurement system 3000 that may be employed to implement processing functionality for various aspects of the invention (e.g., as a transmitter, receiver, processor, memory device, and so on). Those skilled in the relevant art will also recognize how to implement the invention using other computer systems or architectures. Measurement system 3000 may represent, for example, a desktop, mainframe, server, memory device, mobile client device, or any other type of special or general purpose computing device as may be desirable or appropriate for a given application or environment. Measurement system 3000 can include one or more processors, such as a processor 504. Processor 504 can be implemented using a general or special purpose processing engine such as, for example, a microprocessor, microcontroller or other control logic. In this example, processor 504 is connected to a bus 502 or other communication medium.

Measurement system 3000 can also include a main memory 508, for example random access memory (RAM) or other dynamic memory, for storing information and instructions to be executed by processor 504. Main memory 508 also may be used for storing temporary variables or other intermediate information during execution of instructions to be executed by processor 504. Measurement system 3000 may likewise include a read only memory (“ROM”) or other static storage device coupled to bus 502 for storing static information and instructions for processor 504.

The measurement system 3000 may also include information storage mechanism 510, which may include, for example, a media drive 512 and a removable storage interface 520. The media drive 512 may include a drive or other mechanism to support fixed or removable storage media, such as a hard disk drive, a floppy disk drive, a magnetic tape drive, an optical disk drive, a CD or DVD drive (R or RW), or other removable or fixed media drive. Storage media 518 may include, for example, a hard disk, floppy disk, magnetic tape, optical disk, CD or DVD, or other fixed or removable medium that is read by and written to by media drive 514. As these examples illustrate, the storage media 518 may include a computer-readable storage medium having stored therein particular computer software or data.

In alternative embodiments, information storage mechanism 510 may include other similar instrumentalities for allowing computer programs or other instructions or data to be loaded into measurement system 3000. Such instrumentalities may include, for example, a removable storage unit 522 and an interface 520, such as a program cartridge and cartridge interface, a removable memory (for example, a flash memory or other removable memory module) and memory slot, and other removable storage units 522 and interfaces 520 that allow software and data to be transferred from the removable storage unit 518 to measurement system 3000.

Measurement system 3000 can also include a communications interface 524. Communications interface 524 can be used to allow software and data to be transferred between measurement system 3000 and external devices. Examples of communications interface 524 can include a modem, a network interface (such as an Ethernet or other NIC card), a communications port (such as for example, a USB port), a PCMCIA slot and card, etc. Software and data transferred via communications interface 524 are in the form of signals which can be electronic, electromagnetic, optical, or other signals capable of being received by communications interface 524. These signals are provided to communications interface 524 via a channel 528. This channel 528 may carry signals and may be implemented using a wireless medium, wire or cable, fiber optics, or other communications medium. Some examples of a channel include a phone line, a cellular phone link, an RF link, a network interface, a local or wide area network, and other communications channels.

In this document, the terms “computer program product” and “computer-readable medium” may be used generally to refer to media such as, for example, memory 508, storage device 518, and storage unit 522. These and other forms of computer-readable media may be involved in providing one or more sequences of one or more instructions to processor 504 for execution. Such instructions, generally referred to as “computer program code” (which may be grouped in the form of computer programs or other groupings), when executed, enable the measurement system 3000 to perform features or functions of embodiments of the present invention.

In an embodiment where the elements are implemented using software, the software may be stored in a computer-readable medium and loaded into measurement system 3000 using, for example, removable storage drive 514, drive 512 or communications interface 524. The control logic (in this example, software instructions or computer program code), when executed by the processor 504, causes the processor 504 to perform the functions of the invention as described herein.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention. Moreover, aspects of the invention describe in connection with an embodiment may stand alone as an invention.

Furthermore, although individually listed, a plurality of means, elements or method steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

Moreover, it will be appreciated that various modifications and alterations may be made by those skilled in the art without departing from the spirit and scope of the invention. The invention is not to be limited by the foregoing illustrative details, but is to be defined according to the claims.

What is claimed is:

1. Apparatus for Doppler sensing of physiological motion of at least one subject, the apparatus comprising:
a quadrature receiver for receiving a source signal and a modulated source signal, the modulated source signal associated with a transmitted signal reflected from at least one subject; and
logic for mixing the source signal and the modulated signal to generate I and Q data.

2. The apparatus of claim 1, further comprising for non-linear demodulation of the received modulated source signal.

3. The apparatus of claim 2, further comprising logic for arctangent demodulation of the I and Q data.

4. The apparatus of claim 1, further comprising logic for demodulating the I and Q channel data to remove a null.

5. The apparatus of claim 1, further comprising logic for removing DC offset from the I and Q data.

6. The apparatus of claim 1, further comprising logic for center tracking compensation.

7. The apparatus of claim 6, further comprising logic for extracting information associated with an arc from the received signal and estimating a center of the arc.

8. The apparatus of claim 7, wherein the information is rotated prior to estimating the center of the arc.

9. The apparatus of claim 7, where the center of the arc is determined for each pair of points, and the results combined to get an improved estimate of the center.

10. The apparatus of claim 9, wherein the logic is the median.

11. The apparatus of claim 1, further comprising logic for compensating for a phase and amplitude imbalance factor.

12. The apparatus of claim 1, further comprising a phase shifter for introducing a local oscillator signal, and determining phase and amplitude imbalance between the received signal and the local oscillator signal.

13. The apparatus of claim 1, further comprising a voltage controlled oscillator for providing both the transmitted source signal and a local oscillator signal, wherein the local oscillator signal is further divided to provide two orthonormal baseband signals.

14. The apparatus of claim 1, wherein the quadrature receiver is analog.

15. The apparatus of claim 1, wherein the quadrature receiver is digital.

16. The apparatus of claim 1, further comprising logic for linear demodulation of the received modulated source signal.

17. The apparatus of claim 16, wherein the linear demodulation is a singular value decomposition.

18. The apparatus of claim 1, further comprising logic for performing a blind source separation process to isolate one of multiple subjects.

19. A data acquisition system for Doppler radar sensing of physiological motion of at least one subject, the data acquisition apparatus comprising:
an analog to digital converter; and
an automatic gain control unit, wherein the analog to digital converter and the automatic gain control unit are configured to increase the dynamic range of the system, one or both of modifying the DC offset value and gain for the signal of interest.

20. The data acquisition system of claim 19, wherein modifying the DC offset value comprises removing the DC offset.

21. The data acquisition system of claim 19, wherein modifying the DC offset value comprises removing the DC offset, and adjusting and recording the gain.

22. The data acquisition system of claim 19, further comprising tracking and removing a DC offset value.

23. The data acquisition system of claim 19, wherein modifying the DC offset value comprises removing and recording the DC offset, and adjusting and recording the gain.

24. The data acquisition system of claim 19, further comprising adjusting and recording the gain.

25. The data acquisition system of claim 19, further comprising a first analog to digital converter and a DAC for acquiring a DC offset value and outputting a reference.

26. The data acquisition system of claim 25, further comprising a VGA and a second analog to digital converter for providing feedback for the automatic gain control unit.

27. The data acquisition system of claim 19, further comprising logic for performing arctangent demodulation of the received signals.

28. A method for detecting presence and/or sensing physiological motion of at least one subject with a quadrature Doppler receiver, the method comprising the acts of:
receiving a source signal and a modulated source signal, the modulated source signal associated with a transmitted signal reflected from none or at least one subject; and
mixing the source signal and the modulated signal to generate I and Q data.

29. The method of claim 28, further comprising non-linearly demodulating the received modulated source signal.

30. The method of claim 29, further comprising combining the I and Q data using arctangent demodulation.

31. The method of claim 28, further comprising combining the I and Q data using arctangent demodulation and DC offset compensation.

32. The method of claim 28, further comprising compensating for a phase and amplitude imbalance factor of the analog quadrature receiver.

33. The method of claim 28, further comprising introducing a local oscillator signal and determining phase and amplitude imbalance between two analog quadrature receiver chains.

34. The method of claim 28, further comprising providing the transmitted source signal and a local oscillator signal to the receiver, wherein the local oscillator signal is further divided to provide two orthonormal baseband signals.

35. The method of claim 28, further comprising linearly demodulating the received modulated source signal.

36. The method of claim 35, wherein the linear demodulation is a singular value decomposition.

37. The method of claim 28, further comprising performing a blind source separation process to isolate one of multiple subjects.

38. The method of claim 28, further comprising providing the transmitted source signal and a local oscillator signal to the receiver, wherein the quadrature mixing is performed in digital domain.

39. A computer program product comprising computer-readable program code for sensing physiological motion of a subject in a Doppler radar system, the product comprising program code for:
determining presence and/or physiological motion associated with at least one subject from I and Q data output from a quadrature receiver, the I and Q data based on a source signal and a modulated source signal having been modified by at least one subject.

40. The computer program product of claim 39, further comprising program code for combining the I and Q data using arctangent demodulation.

41. The computer program product of claim 39, further comprising program code for combining the I and Q data using arctangent demodulation and DC offset compensation.

42. The computer program product of claim 39, further comprising program code for compensating for a phase and amplitude imbalance factor of a receiver.

43. The computer program product of claim 39, further comprising program code for linearly demodulating the received modulated source signal.

44. The computer program product of claim 39, further comprising program code for non-linearly demodulating the received modulated source signal.

45. The computer program product of claim 38, wherein the linear demodulation is a singular value decomposition.

46. The computer program product of claim 39, further comprising program code for a blind source separation process to isolate one of multiple subjects.
